(19) United States
(12) Patent Application Publication
(10) Pub. No.: US 2014/0335172 A1
(43) Pub. Date: Nov. 13, 2014

(54) OPHTHALMOLOGICAL RINSING AGENT AND METHODS THEREFOF

(71) Applicant: THE UNIVERSITY OF HONG KONG, Hong Kong (CN)

(72) Inventors: Ho Cheung SHUM, Kowloon (HK); Sai Hung WONG, Belair (HK); Yau Kei CHAN, New Territories (HK)

(73) Assignee: THE UNIVERSITY OF HONG KONG, Hong Kong (CN)

(21) Appl. No.: 14/264,869

(22) Filed: Apr. 29, 2014

Related U.S. Application Data
(60) Provisional application No. 61/822,219, filed on May 10, 2013.

(51) Int. Cl. A61K 31/695

(52) U.S. Cl. CPC A61K 31/695
USPC 424/455; 514/63

(57) ABSTRACT

Disclosed herein is a novel rinsing agent, which may be suitable for use in surgical, and, in particular, ophthalmological procedures. The rinsing agent may by employed to remove emulsified oil droplets from the eye cavity. Also disclosed is a method for effectively removing emulsified silicone oil droplets remaining in the eye cavity after ophthalmological procedures using the disclosed rinsing agent in order to prevent the development of long-term complications associated with the emulsified oil droplets.

### RELATED APPLICATIONS

This application claims the benefit of U.S. provisional application Ser. No. 61/822,219, filed May 10, 2013, which is hereby incorporated by reference in its entirety.

### 1. INTRODUCTION

Disclosed herein is a rinsing agent. In certain embodiment, it is a rinsing agent for surgical operations. In certain embodiment, it is used following an ophthalmological procedure. Also disclosed is a low-molecular-weight silicone oil with or without hydrophobic surfactants for use as a rinsing agent. In certain embodiment, provided herein is a method for rinsing an eye cavity to reduce the build-up of silicone oil following ophthalmological procedures using the disclosed rinsing agent. In certain embodiments, the disclosed agent is effective for reducing complications associated with the emulsification of silicone oil in the eye cavity during ophthalmological procedures.

### 2. BACKGROUND

Many diseases found in the human eyes may be attributed to pathophysiological changes in the retina or in the vitreous body. The vitreous body occupies a major portion of a volume of the eye and may serve to maintain the shape of the eye. The vitreous body may be located near the center of the eye, posterior of the lens and anterior of the retina. The vitreous body may comprise approximately 98.0% water and approximately 2.0% of hyaluronic acid. A network of collagen fibers may also be present to stabilize the water content at least partially due to the water retention ability of the collagen fibers. This may result in formation of its gelatinous structure, which may protect surrounding structures and tissues against mechanical trauma and maintains positions of the lens and retina.

Methods employed for treating eye disorders of the retina, including retinal detachment and proliferative vitreoretinopathy (PVR), have increased considerably in recent years. An increase may be at least partially in response to the rapid advancement of surgical techniques in removing the vitreous body (vitrectomy). For example, a pars plana vitrectomy (PPV) is a surgical procedure that involves removal of a vitreous gel from the eye. PPV was a major advancement because for the first time it allowed for the removal of vitreous through a closed system, rather than through an open sky technique. Additionally, a three-port PPV technique has been developed to remove the vitreous by making three small incisions in the sclera of the eye and extending to the posterior part of the eye where the vitreous body is located. Small gauge instruments may be introduced through the small incisions; considerable portion of the vitreous can be removed efficiently. Three-port PPV comprises of fiberoptic light, infusion cannula and vitreous cutter; it has become the gold standard and has remained so for at least 3 decades. Three-port PPV is widely used to correct visually devastating conditions including retinal detachments, macular degeneration, and diabetic eye diseases. After the PPV procedure, an intraocular tamponade is used for facilitating the retinal reattachment. Combined with vitreoretinal surgery, silicone oil injection has become a standard technique and improves the surgical outcomes of complex retinal detachment associated with proliferative vitreoretinopathy, giant retinal tears, proliferative diabetic retinopathy, or ocular trauma. Usually silicone oil with a dynamic viscosity 1000 mPa·s and 5000 mPa·s of are used for retinal tamponade.

However, if silicone oil is not effectively removed following the procedure, it can lead to long-term complications, particularly cataract, glaucoma, and keratopathy, due to its emulsification with the surrounding body fluid. In current clinical practice, a kind of sterilized saline, e.g., balanced salt solution (BSS), which mimics the intraocular fluid, is used to rinse the eye cavity after the removal of the bulk silicone oil. However, this approach has proven to be ineffective.

Thus, there is a need for a rinsing agent that can effectively remove emulsified silicone oil droplets, thereby reducing complications associated with the emulsification of silicone oil during ophthalmic procedures.

### 3. SUMMARY

Provided herein is a rinsing agent, effective for the removing emulsified silicone oil droplets remaining in the eye cavity after ophthalmological procedures in order to prevent the development of long-term complications. The rinsing agent provided herein comprising a silicone oil with a certain density, viscosity and surface tension. In certain embodiments, the silicone oil is a low-molecular-weight silicone oil. In certain embodiments, the silicone oil is a high-molecular-weight silicone oil. In certain embodiments, the silicone oil has a high volatility. In certain embodiments, the silicone oil has a dynamic viscosity of approximately 0.5 mPa·s to approximately 10.0 mPa·s. In certain embodiments, the silicone oil has low volatility. In certain embodiments, the silicone oil in the rinsing agent is Hexamethyldisiloxane. In another embodiment, the silicone oil in the rinsing agent is Octamethyltrisiloxane. In certain embodiments, the rinsing agent has a v/v ratio of silicone oil of 0.5-1%, 1-10%, 10-20%, 20-30%, 30-40%, 40-50%, 50-60%, 60-70%, 70-80% 80-90%, or 90-99%.

In certain embodiments described herein, the rinsing agent also consists of at least one active ingredient that is a biocompatible hydrophobic surfactant, which can be used to create a double emulsion to enhance the removal of emulsified oil droplets from the eye cavity. In certain embodiments, the rinsing agent has a v/v ratio of biocompatible hydrophobic surfactant of approximately 0.5% to approximately 20%. The presence of a hydrophobic surfactant in the rinsing agent results, at least in part, in an oil-in-water-in-oil double emulsion, where the emulsified silicone oil droplets are encapsulated by aqueous globules. The hydrophobic surfactant in the rinsing agent at least partially stabilizes said aqueous globules encapsulated with emulsified silicone oil droplets within said rinsing agent. In certain embodiments provided herein, the hydrophobic surfactant is a blend of approximately 50.0% trimethylsiloxysilicate and approximately 50.0% cyclomethicone (decamethylpenta-cyclosiloxane).

Provided herein is a method for effectively removing emulsified silicone oil droplets remaining in the eye cavity after ophthalmological procedures to prevent the development of long-term complications. The rinsing agent can be administered during or after a surgery or procedure. In one or more embodiments, the method comprises initiating a surgical procedure, and rinsing the eye cavity with the rinsing agent. In certain embodiments, the rinsing agent can be administered to the eye of a subject in the following manner: 1) after the removal of the bulk silicone oil after the surgical procedure, emulsified silicone oil droplets (in the form of oil-in-water emulsion) may remain; 2) a relatively large amount of the rinsing agent is mixed with the oil-in-water emulsion in the eye cavity to trigger the formation of the o/w/o double emulsion due to the presence of hydrophobic surfactant in the rinsing agent; 3) unwanted emulsified silicone oil droplets are encapsulated by the larger aqueous globules; 4) the aqueous globules encapsulated with emulsified oil droplets gathering at a bottom portion of the eye cavity as a result of a higher density of aqueous solution than rinsing agent; 5) the doubly emulsified droplets may be removed by suction and by repeatedly permitting an air/rinsing agent exchange; and 6) a surgical long-acting gas may be used to fill up the eye cavity at the end of the operation after the rinsing agent has been removed.

In one or more embodiments, the rinsing agent may further consist of other therapeutic agents, including proteins and proteinaceous compounds, steroids, nonsteroidal anti-inflammatories, antibacterials, anti-infective agents, antivirals, antifungals, insulins, Glugacon-like Peptide-1 and its analogs, Beta Adrenergic Blocking Agents, antihistamines, anti-microtubule agents, vitrectomy agents, therapeutic antibodies, anti-glaucoma agents, buffering agents, tonicity agents, viscosity enhancing agents, preservatives, and healing agents.

### 4. DESCRIPTION OF THE FIGURES

FIG. 1. Schematic diagram of the occurrence of emulsification (oil-in-water emulsion formation) after using the silicone oil as the intraocular tamponade in the eye cavity.

FIG. 2. Diagram showing the interaction of surfactants within an oil-in-water emulsion.

FIG. 3. Diagram identifying phases of a water-in-oil emulsion and an oil-in-water emulsion.

FIG. 4. Schematic diagram identifying steps in a process for using saline as a rinsing agent.

FIG. 5. Diagram comparing the use of silicone oil (1.0 cSt, 0.65 cSt) versus other rinsing agents in the eye cavity.

FIG. 6. Diagram showing the conversion of an oil-in-water emulsion to an oil-in-water-in-oil double emulsion via the addition of a rinsing agent.

FIG. 7. Schematic diagram of the separation of the oil and aqueous phase of an emulsion.

FIG. 8. Schematic diagram showing that a low-MW silicone oil combined with a hydrophobic surfactant (emulsifier) and an oil-in-water emulsion produces an oil-water-in-oil emulsion.

FIGS. 9A-B. Diagrams illustrating that the double emulsion is much larger in size (B) compared with a typical emulsion (A).

FIGS. 10A-E. Diagrams illustrating the method of using and ophthalmological rinsing agent according to at least one embodiment.

FIGS. 11A-B. Silicone oil with fluorescent labeling. (A) Silicone oil before labeling (Left) and after BODIPY labeling (Right). (B) Silicone oil labeled with BODIPY remains transparent.

FIGS. 12A-C. Microscopic images of an oil-in-water emulsion. Bright field (A), BODIPY labeling (B) and the merged image (C) of A and B. Magnification: 100×. The scale bars are 100 μm.

FIGS. 13A-B. Microscopic images of double emulsion focusing on (A) outer boundaries and (B) inner boundaries. In (B), small droplets are clearly shown which were encapsulated by the bigger bubbles. Magnification: 100×. The scale bars are 100 μm.

FIG. 14A-C. Microscopic images of oil-in-water-in-oil double emulsion. Bright field (A), BODIPY labeling (B) and the merged image (C) of A and B. Magnification: 100×. The scale bars are 100 μm.

FIG. 15. Schematic diagram of the method of the in vitro eye model rinsing experiment discussed in section 6.2.

FIG. 16. Amount of emulsified droplets remaining in the chamber after washing. The value of rinsing agent has been normalized with respect to the value of BSS group (t-test, ***, p<0.001; Error bar=±SD; n=8).

FIG. 17. Diagram of the in vitro culture test discussed in section 6.4.

FIG. 18. Cell viability (relative to HBSS) of RGC-5 after incubating with the testing agent for 1 hour using MTS assay. (One-way ANOVA followed by Bonferroni post test, Error bar=±SD; n=3)

FIG. 19. Percentage of dead RGC-5 cells after incubating with the testing agent for 1 hour using LDH assay (One-way ANOVA followed by Bonferroni post test, Error bar=±SD; n=3).

FIGS. 20A-F. Microscopic images of the RGC-5 morphologies after incubating with different types of rinsing agent for 1 hr in vitro.

FIG. 21. Cell viability (relative to HBSS) of rMC-1 cells after incubating with the testing agent for 1 hour using MTS assay (One-way ANOVA followed by Bonferroni post test, Error bar=±SD; n=3).

FIG. 22. Percentage of dead rMC-1 cells after incubating with the testing agent for 1 hour using LDH assay (One-way ANOVA followed by Bonferroni post test, Error bar=±SD; n=3).

FIGS. 23A-F. Microscopic images of the rMC-1 morphologies after incubating with different types of rinsing agent for 1 hour in vitro.

FIG. 24. RGC-5 Cell viability relative to HBSS after exposure to different testing agents for 30 minutes using MTS assay (One-way ANOVA test followed by Bonferroni Multiple Comparison test; *, p<0.05; Error bar=±SD; n=3).

FIG. 25. RGC-5 Cell death after exposure to different testing agents for 30 minutes using LDH assay (One-way ANOVA test followed by Bonferroni Multiple Comparison test; Error bar=±SD; n=3).

Reference is made in the following detailed description to the accompanying drawings, which form a part hereof, wherein like numerals may designate like parts throughout to indicate corresponding or analogous elements. For simplicity and/or clarity of illustration, elements illustrated in the figures have not necessarily been drawn to scale. For example, the dimensions of some of the elements may be exaggerated relative to other elements for clarity. Further, it is to be understood that other embodiments may be utilized and structural and/or logical changes may be made without departing from the scope of the disclosure. It should also be noted that directions and references such as, for example, up, down, top, bottom, over, above and so on, may be used to facilitate the discussion of the drawings and are not intended to restrict application of disclosure. Therefore, the following detailed description is not to be taken in a limiting sense and the scope of the disclosure is intended to be defined by the appended claims and equivalents.

### 4.1 Definitions

When referring to the compounds provided herein, the following terms have the following meanings unless otherwise indicated.

As used herein, the terms “saline,” “saline solution,” “isotonic saline solution,” “sterilized saline,” are interchangeable. Such terms can also refer to specific formulations, including balanced salt solution (BSS), Hank's Balanced Salt Solution (HBSS), and phosphate buffered saline (PBS).

As used herein, the terms “washing” and “rinsing” are interchangeable.

As used herein, silicone oil can be abbreviated as “SO”.

As used herein, the terms “ophthalmic” and “ophthalmological” are used interchangeably.

### 5. DETAILED DESCRIPTION

In the following detailed description, numerous specific details are set forth to provide a thorough understanding of claimed subject matter. However, it will be understood by those skilled in the art that claimed subject matter may be practiced without these specific details. In other instances, methods, apparatuses, or systems that would be known by one of ordinary skill have not been described in detail so as not to obscure claimed subject matter.

Reference throughout this specification to one implementation, an implementation, one embodiment, an embodiment, or the like may mean that a particular feature, structure, or characteristic described in connection with a particular implementation or embodiment may be included in at least one implementation or embodiment of claimed subject matter. Thus, appearances of such phrases in various places throughout this specification are not necessarily intended to refer to the same implementation or to any one particular implementation described. Furthermore, it is to be understood that particular features, structures, or characteristics described may be combined in various ways in one or more implementations. In general, of course, these and other issues may vary with the particular context. Therefore, the particular context of the description or usage of these terms may provide helpful guidance regarding inferences to be drawn for that particular context.

Likewise, the terms, “and,” “and/or,” and “or” as used herein may include a variety of meanings that will, again, depend at least in part upon the context in which these terms are used. Typically, “and/or” as well as “or” if used to associate a list, such as A, B or C, is intended to mean A, B, or C, here used in the exclusive sense, as well as A, B and C. In addition, the term “one or more” as used herein may be used to describe any feature, structure, or characteristic in the singular or may be used to describe some combination of features, structures, or characteristics.

Embodiments of the current application may be useful during and/or responsive to various ophthalmic surgical procedures. In certain embodiment, the procedures are related to those in which the vitreous body is attached to an inner limiting membrane. In certain embodiment, the inner membrane is the innermost layer of the retina. In certain embodiment, as a result of aging, a vitreous body may be found to be liquefied and to shrink. This physiological change of the structure of vitreous may occasionally cause the retina to fall off the back of the eye and may be regarded as one of the leading causes of retinal detachment. Thus, vitrectomy, and in certain embodiment, a pars plana vitrectomy (PPV), has become a standard procedure in treating retinal detachment, such as proliferative vitreoretinopathy (PVR) in order to eliminate the traction to the retina due to the shrinkage of vitreous body, which may induce further retinal detachment or hinder the retinal reattachment process after surgical fixation procedures.

Surgical fixation procedures may be performed after vitrectomy. After a surgical fixation procedure, a substitute for vitreous may be needed to maintain a shape of the eye and to prevent secondary damage which may, at least in part, result from a loss of pressure that may occur after a vitreous body is removed. In certain embodiment, silicone oil is used as an intraocular tamponade. Silicone oil is a liquid, polymerized siloxane with organic side chains. They are formed with a backbone of alternating silicone-oxygen atoms ( . . . Si—O—Si—O—Si . . . )—In certain embodiment, the silicone oil is a straight-chain siloxane. In certain embodiment, the silicone oil is polydimethylsiloxane (PDMS), which has the following structure:

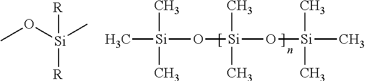

Silicone oil may provide a mechanical support within the eye cavity, and most importantly, at least in some embodiments, may at least partially result in retinal reattachment due to a tamponade effect.

Silicone oil is frequently used in eye surgery when the retina is detaching, a condition common in patients with diabetes. The surgeon fills the eye with silicone oil, and this helps the retina to adhere after surgery.

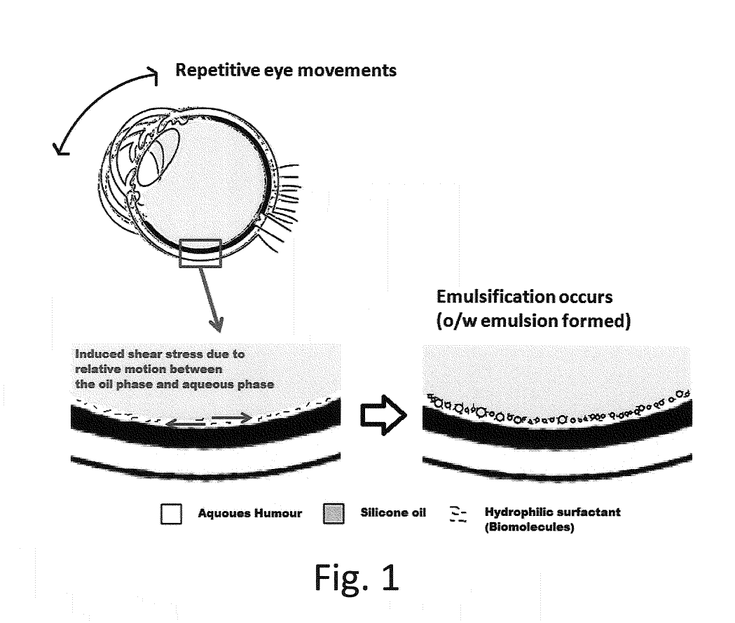

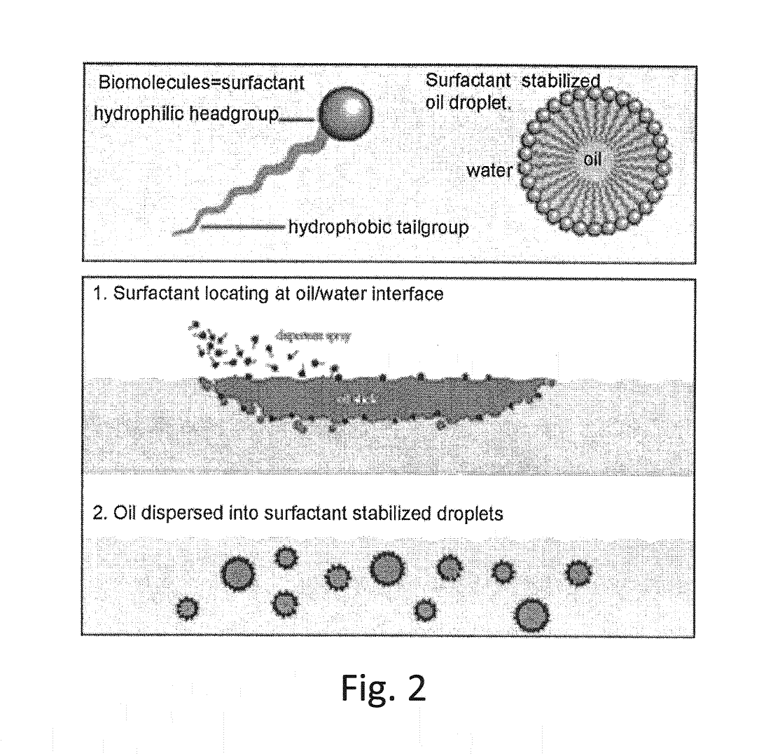

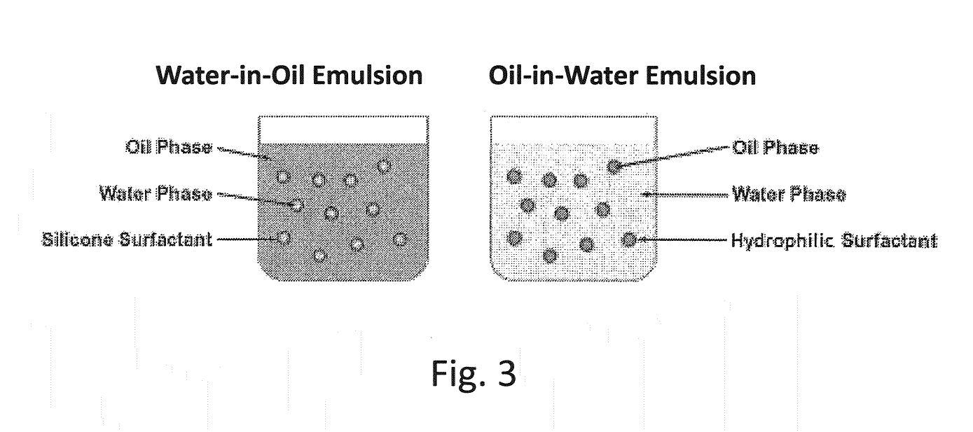

Although silicone oil may be used in assisting the retinal reattachment, there are problems if silicone oil is used as an intraocular tamponade. For example, during eye movements, as injected silicone oil maintains no or, perhaps, only a minimal coupling to the eye wall, there may often be relative motion between the silicone oil and the aqueous humor, and between the aqueous humor and the eye wall. In certain embodiments, relative motion imposes a parallel shear force, which acts on the interface between the aqueous humor and the eye wall. This force scrapes surface molecules from the bulk silicone oil phase, which forms tiny silicone oil droplets. The tiny oil droplets are stabilized by biomolecules, such as amino acids present in the aqueous humor, and become stable and remain in the aqueous phase (FIG. 1 and FIG. 2). This phenomenon, which may be referred to as emulsification, and may give rise to an oil-in-water (o/w) emulsion formed, meaning that an oil phase may be dispersed in a water phase and comprise droplets (FIG. 3). In contrast, a water-in-oil (w/o) emulsion is one in which a water phase may be dispersed in an oil phase and comprise droplets (FIG. 3). These tiny oil droplets ideally should coalesce with the bulk of the silicone oil phase; however, due to the presence of some surface active biomolecules such as proteins (amino acids) or phospholipids (also regarded as a hydrophilic surfactant), the tiny oil droplets are stabilized. Emulsification may be regarded as a major reason for postoperative complications in the eye, and can be related to use of silicone oil as the intraocular tamponade. Silicone oil vacuoles have been found in many places within the eye cavity, such as within the cornea structure, in the anterior chamber, in the trabecular meshwork, at the lens capsule, in the sub retinal space, as well as in the optic nerve. Studies have shown that the emulsified droplets presented in these regions may give rise to a number of complications, such as cataracts, glaucoma, inflammation, and PVR. Other complications such as open-angle closure glaucoma are also caused by an emulsification of silicone oil.

To avoid further emulsification thus generating much more emulsified droplets within the eye cavity, the bulk silicone oil is recommended to be removed after the retina is confirmed to be reattached or if emulsification is identified. In certain embodiments, resulting at least in part from silicone oil emulsification, a silicone oil intraocular tamponade is recommended for removal approximately 3 to 6 months after ophthalmic procedure or after the retina is confirmed to be reattached. However, in certain instances, the removal of silicone oil may not guarantee avoidance of complications. Incidence of silicone oil induced glaucoma is high. Additionally, silicone oil induced glaucoma comprises a progressive condition that brings about a need for life-long follow up, one or more hospital visits, as well as costly and/or complex treatment.

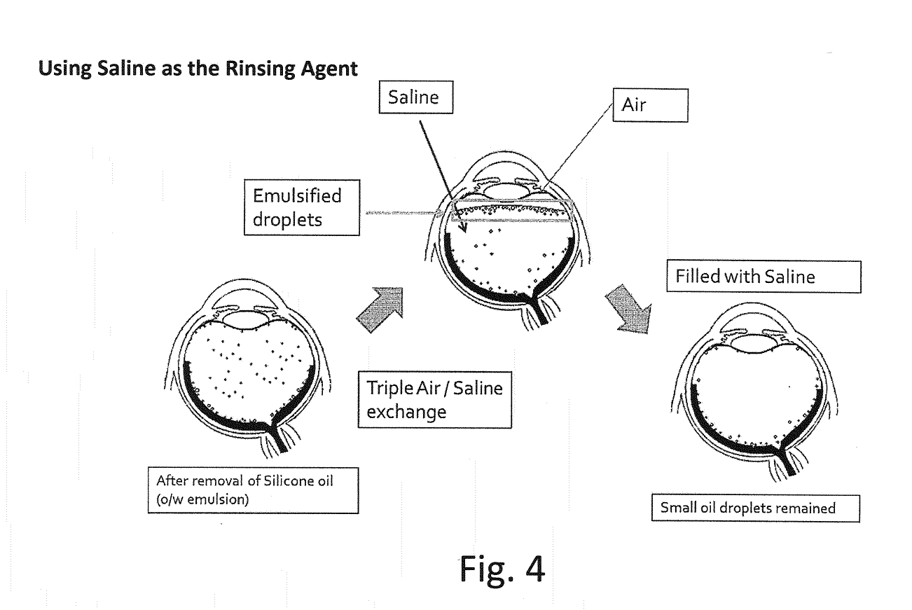

In current clinical practice, an isotonic saline solution such as balanced salt solution (BSS), which mimics the intraocular fluid, is used as a rinsing agent to rinse the eye cavity after the removal of silicone oil as a means of flushing emulsified silicone oil droplets from the eye cavity (FIG. 4). A typical removal procedure is similar to three-port PPV. This time, the vitreous cutter is used to aspirate silicone oil and infusion cannula is used to perform air/fluid exchange to flush the eye cavity and remove the remaining silicone oil.

However, a frequently used isotonic saline solution is not capable of adequately removing emulsified oil droplets from the eye cavity. Accordingly, after rinsing, residual droplets are still present inside the eye cavity. In some instances, even after two or three repeated rinsing operations, droplets are still present in the eye cavity. The continuing presence of droplets in the eye cavity is attributed to the emulsified oil droplets being small, stable, and buoyancy neutral. In other words, these emulsified droplets are very stable in the intraocular fluid, which is aqueous in nature. Further, an interface of the droplets may be stabilized by a layer of surface-active biomolecules, which renders droplets adherent to biological tissues (e.g., intraocular tissue) under many circumstances. Thus, rinsing is not sufficient to dislodge droplets from the eye cavity. Although rinsing by the isotonic saline solution may dilute the o/w emulsion, the solution is not capable of completely removing the emulsified droplets.

Thus, in certain embodiments, a rinsing agent is provided that surpasses the ability of isotonic saline solutions to function as a rinsing agent for removing emulsified oil droplets. In certain embodiments, a rinsing agent is provided for use by surgeons and other medical professionals to remove oil droplets during and/or after a surgical procedure. In certain embodiments, the rinsing agent is easily obtained and injected, and is physiologically acceptable and well tolerated in transient use. In certain embodiments, the rinsing agent is physiological acceptable and easy to introduce while permitting excellent visibility within the eye cavity for surgical use in ophthalmological procedures. Such visibility enhances the use of certain surgical devices. In certain embodiments, the surgical uses are vitrectomies and related procedures. In certain embodiments, the rinsing agent is used in removing unwanted emulsified silicone oil droplets efficiently in a manner that prevents postoperative complications associated with emulsification of silicone oil intraocular tamponade.

It has been found that it may be advantageous for surgical operations in eyes to use a non-aqueous and water-immiscible as an intraoperative tool. For example, in United States published patent application 2010/0274215, a low-molecular weight silicone oil as a conventional ophthalmology fluid for flushing during a vitrectomy is described. Such an ophthalmology fluid does not mix with blood, thus preventing clouding and impairment of visibility if hemorrhage occurs.

In certain embodiments, double emulsion systems are used as a rinsing agent due to the advantage offered by the core-shell structures. There are two main types of double emulsions: (i) water-in-oil-in-water type (w/o/w) where water droplets are dispersed in oil droplets, which in turn are dispersed in a continuous aqueous phase, and (ii) oil-in-water-in-oil type (o/w/o) where oil droplets are located within water droplets that are dispersed within continuous oil. These emulsions are with dispersed oil globules containing smaller aqueous droplets or vice versa. An application of the w/o/w system has been applied in human pharmaceuticals as drug delivery systems for carrying hydrophilic drugs to target tissues.

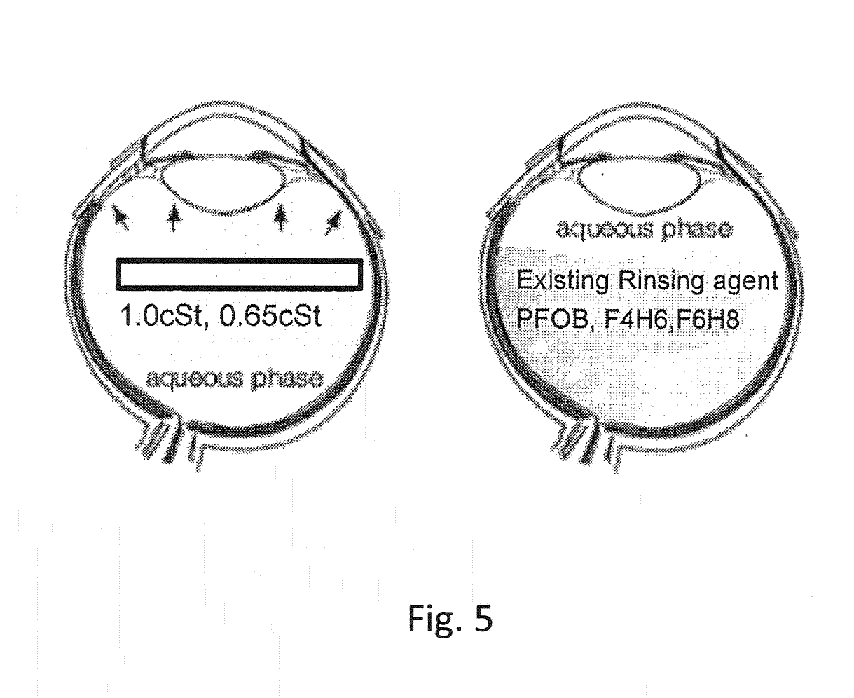

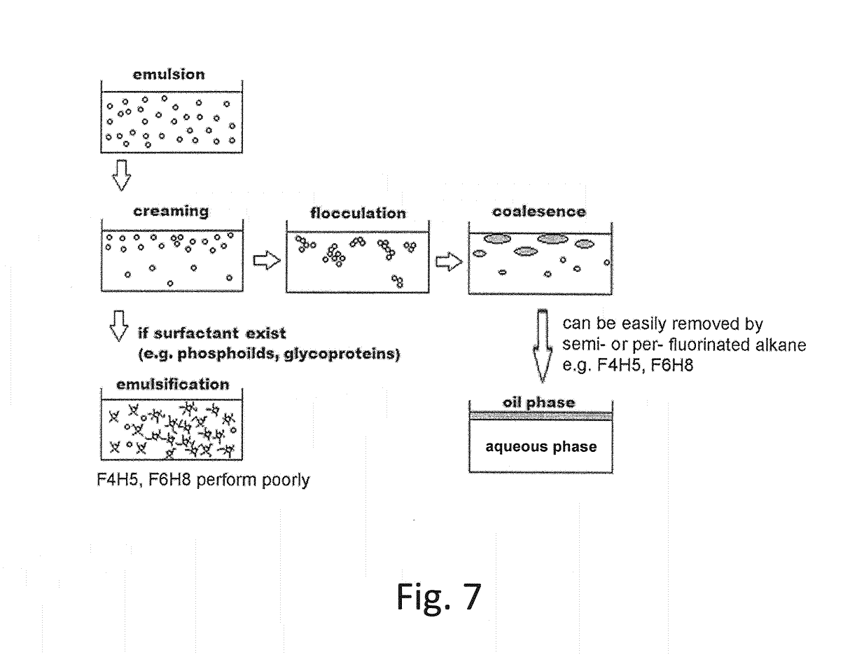

After silicone oil emulsification occurs within the eye cavity, the oil droplets disperse stably within the aqueous phase and cannot be efficiently removed by its solvent, since oil solvents such as F4H5 and F6H8 are immiscible with water (FIGS. 5 and 7).

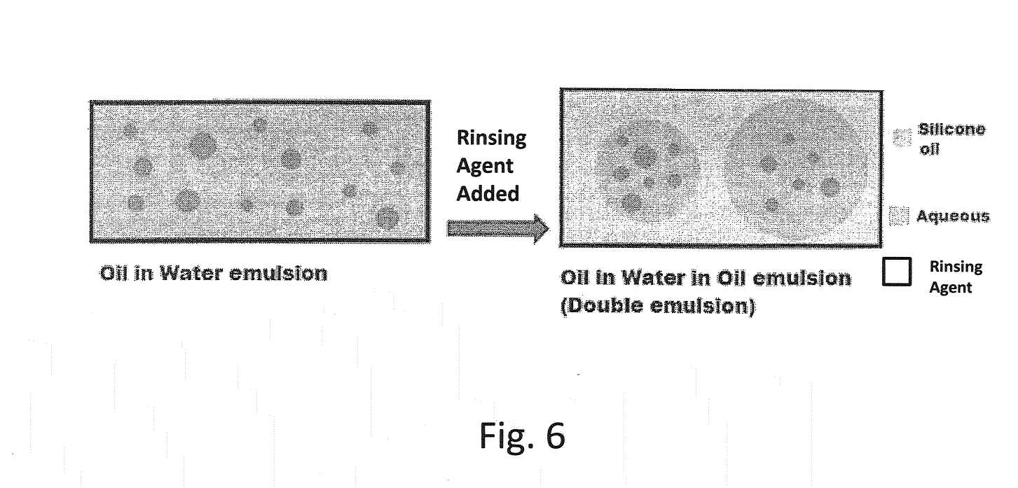

As shown in FIG. 6, an application of the oil-in-water-in oil emulsion comprises to enlarge the size of oil droplets. After an additional step of emulsification to form double emulsion, the silicone oil-water emulsion is further encapsulated by the continuous low molecular weight (MW) oil phase. Since the low MW oil phase is lighter than water, it can be removed more easily and efficiently together with the silicone oil droplets (FIG. 7).

A rinsing agent that accords with an embodiment may have the effect including little or negligible mixing with intraocular fluid and blood. Accordingly, visibility of the material may be unaffected by the presence of such fluids, for example. Moreover, a rinsing agent may employ an o/w/o double emulsion system during use for removing emulsified silicone oil droplets by way of the ability to transport unwanted oil droplets by encapsulating oil droplets within the water globules. The o/w/o double emulsion system is particularly important as it significantly improves efficiency in removing unwanted silicone oil emulsified droplets.

In one embodiment, the rinsing agent is a fluid mixture having a low-MW silicone oil and a biocompatible hydrophobic surfactant for the eye cavity during and/or subsequent to a surgical procedure. Specifically, in at least one embodiment, a rinsing agent for removing silicone oil/emulsified oil droplets in ophthalmic surgery comprises of a low-MW silicone oil, such as Hexamethyldisiloxane 0.65 cSt or Octamethyltrisiloxane, and a hydrophobic surfactant, such as Dow Corning 749 fluids (Cyclopentasiloxane and Trimethylsiloxysilicate=1:1). In one or more embodiments in which the silicone oil is of low MW, the silicone oil has a MW range of 160 g/mol to 170 g/mol, 170 g/mol to 180 g/mol, 180 g/mol to 190 g/mol, 190 g/mol to 200 g/mol, 200 g/mol to 210 g/mol, 210 g/mol to 220 g/mol, 220 g/mol to 230 g/mol, and 240 g/mol to 250 g/mol.

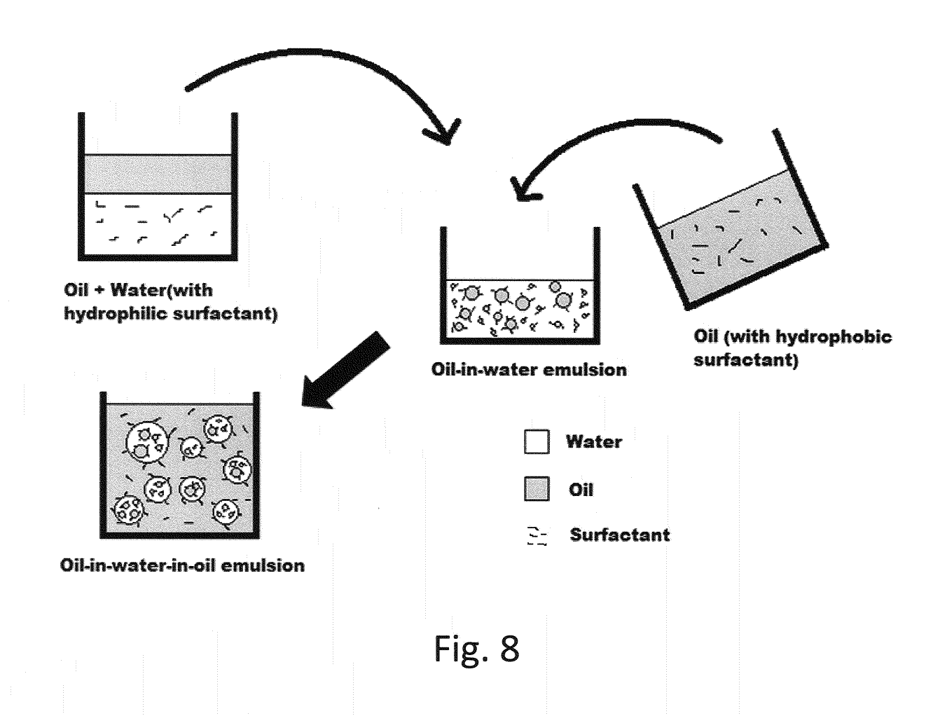

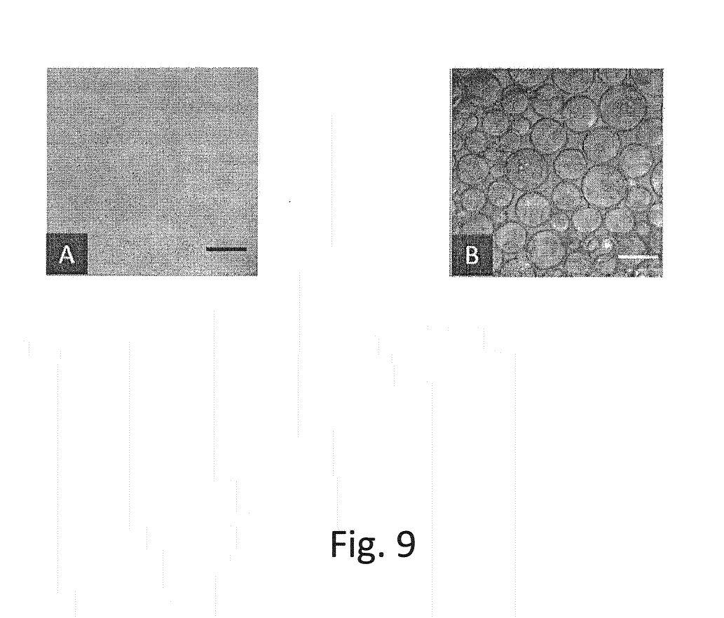

A guiding principle is that a low-MW silicone oil combined with a hydrophobic surfactant (emulsifier) and a w/o emulsion produces an oil-water-in-oil emulsion (FIG. 8). In certain embodiments, the rinsing agent is designed to assist in the removal of the residual silicone oil (i.e., o/w emulsion) remaining in the eye cavity. Specifically, the addition of hydrophobic surfactant reverses the emulsification due to the surface active biomolecules in the eye cavity. Unlike the biomolecules, the hydrophobic surfactants favor the formation of w/o emulsion. After the removal of the high-molecular weight silicone oil intraocular tamponade, a small volume of water content remained in the eye cavity. The infusion of the relative large volume of the rinsing agent with hydrophobic surfactant induces the aqueous content to emulsify as a form w/o emulsion. Since the original emulsified droplets are stabilized in the aqueous fluid, after infusion of the rinsing agent, the aqueous content is forced to emulsify in the outer oil phase. As a result, all these unwanted oil droplets will be encapsulated by the larger water droplets formed during a second emulsification step resulting in a w/o emulsion. The originally tiny silicone-oil droplets will be surrounded by the aqueous content in the form of drops; this is the so-called oil-in-water-in-oil (o/w/o) double emulsion. With a suitable amount of hydrophobic surfactant, this double emulsion results to be very stable. And more importantly, since all the aqueous content within the eye cavity is forced to be emulsified as water-in-oil emulsion, all the emulsified droplets should be captured within the double emulsion (FIG. 8). The o/w/o emulsion is much larger in size with a lower viscosity, thus it is much easier to remove (FIG. 9).

Silicone oils, which are used in association with various embodiments, have been applied in as antifoams and coatings different industrial sectors. In cosmetics, for example, silicone oils are used as glossing agents and agents for improving adhesion. Silicone oils are used in plastic surgeries.

Processes for manufacturing silicone oils are well known; therefore, a desired configuration of silicone oils suitable for a particular field of application is easily and economically synthesized. For example, aliphatic silicones, which include polydimethysiloxane (PDMS), are synthesized and/or obtained relatively inexpensively. The PDMS family of silicone oils extends from low molecular weight to high molecular weight, where the high molecular weight types with a dynamic viscosity range from 1000 mPa·s to 5500 mPa·s are widely used as the intraocular tamponade. In certain embodiments, the silicone oil has a dynamic viscosity range from 1000 mPa·s to 1500 mPa·s, 1500 mPa·s to 2000 mPa·s, 2500 mPa·s to 2500 mPa·s, 3000 mPa·s to 3500 mPa·s, 4000 mPa·s to 4500 mPa·s, 4500 mPa·s to 5000 mPa·s, and 5000 mPa·s to 5500 mPa·s. In other embodiments, the silicone oil has a dynamic viscosity range from 0 mPa·s to 0.5 mPa·s, 0.5 mPa·s to 1.0 mPa·s, 1.5 mPa·s to 2.0 mPa·s, 2.0 mPa·s to 2.5 mPa·s, 2.5 mPa·s to 3.0 mPa·s, 3.0 mPa·s to 3.5 mPa·s, 3.5 mPa·s to 4.0 mPa·s, 4.0 mPa·s to 4.5 mPa·s, 4.5 mPa·s to 5.0 mPa·s, 5.0 mPa·s to 5.5 mPa·s, 5.5 mPa·s to 6.0 mPa·s, 6.0 mPa·s to 6.5 mPa·s, 6.5 mPa·s to 7.0 mPa·s, 7.0 mPa·s to 7.5 mPa·s, 7.5 mPa·s to 8.0 mPa·s, 8.0 mPa·s to 8.5 mPa·s, 8.5 mPa·s to 9.0 mPa·s, 9.0 mPa·s to 9.5 mPa·s, and 9.5 mPa·s to 10.0 mPa·s. Silicone oils with specific ranges of viscosity are highly suitable as a rinsing agent for surgical, in particular ophthalmological operations.

In certain embodiments, the silicone oils have certain viscosity. If viscosity is sufficiently low, a rinsing agent is infused and removed via thin cannulas or needles. In certain embodiments, the dynamic viscosity does not exceed a value of approximately 10.0 mPa·s when measured at 25° C. and ambient pressure using capillary viscometer, such as a Schott CT52 UBBELOHDE. Silicone oils having a dynamic viscosity that does not exceed 10.0 mPa·s when measured at 25° C. are easier to use in ophthalmological surgeries. These silicone oils are more easily infused to the inside of the eye, and can be removed through thin cannulas in corresponding surgical operations. A rinsing agent is applied through up to 25-gauge cannulas, which are typically used in ophthalmological operations.

In certain embodiments, the silicone oil has a kinematic viscosity range from 0 cSt to 0.5 cSt, 0.5 cSt to 1.0 cSt, 1.5 cSt to 2.0 cSt, 2.0 cSt to 2.5 cSt, 2.5 cSt to 3.0 cSt, 3.0 cSt to 3.5 cSt, 3.5 cSt to 4.0 cSt, 4.0 cSt to 4.5 cSt, 4.5 cSt to 5.0 cSt, 5.0 cSt to 5.5 cSt, 5.5 cSt to 6.0 cSt, 6.0 cSt to 6.5 cSt, 6.5 cSt to 7.0 cSt, 7.0 cSt to 7.5 cSt, 7.5 cSt to 8.0 cSt, 8.0 cSt to 8.5 cSt, 8.5 cSt to 9.0 cSt, 9.0 cSt to 9.5 cSt, 9.5 cSt to 10.0 cSt, 10.0 cSt to 10.5 cSt, 10.5 cSt to 11.0 cSt, 11.0 cSt to 11.5 cSt, 11.5 cSt to 12.0 cSt, 12.0 cSt to 12.5 cSt, 12.5 cSt to 13.0 cSt, 13.0 cSt to 13.5 cSt, 13.5 cSt to 14.0 cSt, 14.0 cSt to 14.5 cSt, 14.5 cSt to 15.0 cSt, 15.0 cSt to 15.5 cSt, 15.5 cSt to 16.0 cSt, 16.0 cSt to 16.5 cSt, and 16.5 cSt to 17.0 cSt. In other embodiments, the silicone oil has a kinematic viscosity range from 1000 cSt to 1500 cSt, 1500 cSt to 2000 cSt, 2500 cSt to 2500 cSt, 3000 cSt to 3500 cSt, 4000 cSt to 4500 cSt, 4500 cSt to 5000 cSt, 5000 cSt to 5500 cSt, 5500 cSt to 6000 cSt, 6000 cSt to 6500 cSt, 6500 cSt to 7000 cSt, 7000 cSt to 7500 cSt, 7500 cSt to 8000 cSt, 8000 cSt to 8500 cSt, 8500 cSt, 9000 cSt, and 9000 cSt to 9500 cSt.

In certain embodiments, the silicone oils have a certain range of density. In certain embodiments, the silicone oils have a density of less than that of water (<1.0 g/cm³), same as water or higher than water. In certain embodiment, the silicone oils have a density of <1.0 g/cm³. In at least one embodiment, the silicone oil has a density range from 0.60 g/cm³ to 0.65 g/cm³, 0.65 g/cm³ to 0.70 g/cm³, 0.70 g/cm³ to 0.75 g/cm³, 0.75 g/cm³ to 0.80 g/cm³, 0.80 g/cm³ to 0.85 g/cm³, 0.85 g/cm³ to 0.90 g/cm³, 0.90 g/cm³ to 0.95 g/cm³, 0.95 g/cm³ to <1.0 g/cm³. When the density of the silicone is less than that of water, aqueous globules encapsulating emulsified silicone oil droplets after the double emulsion formation gather at the posterior part of the eye cavity due to a higher density than the rinsing agent. In certain embodiments, assembly of these globules partially facilitates removal of the droplets.

In certain embodiment, the silicone oils have low volatility. Low viscosity silicone oils, for example, having a dynamic viscosity lower than approximately 3.0 mPa·s, are very thin, comprise a relatively high vapor pressure, and accordingly high volatility, making it excellent for use. In at least one embodiment, the silicone oil has relatively high vapor pressure (accordingly high volatility) in the following ranges (at 25° C.): 0 Torr to 5 Torr, 5 Torr to 10 Torr, 10 Torr to 15 Torr, 15 Torr to 20 Torr, 20 Torr to 25 Torr, 25 Torr to 30 Torr, 30 Torr to 35 Torr, 35 Torr to 40 Torr, and 40 Torr to 45 Torr.

Another relevant trait for silicone oils in accordance with one or more embodiments is surface tension. A lower surface tension silicone oil may lead to better readiness in enhancing the formation of double emulsions; however, a silicone oil with a surface tension value that is too low may lead to formation of very small emulsion droplets, which can be difficult to remove. In at least one embodiment, the silicone oils have a surface tension range from 14.0 mN/m to 14.5 mN/m, 14.5 mN/m to 15.0 mN/m, 15.0 mN/m to 15.5 mN/m, 15.5 mN/m to 16 mN/m, 16 mN/m to 16.5 mN/m, 16.5 mN/m to 17.0 mN/m, 17.0 mN/m to 17.5 mN/m, 17.5 mN/m to 18.0 mN/m, 18.0 mN/m to 18.5 mN/m, 18.5 mN/m to 19.0 mN/m, 19.0 mN/m to 19.5 mN/m, 19.5 mN/m to 20.0 mN/m, 20.0 mN/m to 20.5 mN/m, 20.5 mN/m to 21.0 mN/m, 21.0 mN/m to 21.5 mN/m, 21.5 mN/m to 22.0 mN/m, 22.0 mN/m to 22.5 mN/m, 22.5 mN/m to 23.0 mN/m, 23.0 mN/m to 23.5 mN/m, 23.5 mN/m to 24.0 mN/m, 24.0 mN/m to 24.5 mN/m, 24.5 mN/m to 25.0 mN/m.

When a rinsing agent is not removed completely through a couple of air/fluid exchange procedures, a small remaining portion of the rinsing agent is vaporized and leave the eye cavity. This prevents undesirable emulsification which is brought about in response to the use of silicone oils. However, sterilization procedures of silicone oils having a dynamic viscosity below 0.1 mPa·s can be problematic. Moreover, in certain embodiments, toxicity is high for the use of silicone oils having only 1.0 siloxane unit, therefore these silicone oils are not suitable. In certain embodiments, the silicone oils have acceptable biocompatibility.

The polymer chain length also has an effect on other properties of the silicone oil (Table 1).

TABLE 1
Properties of Silicone Oil by Polymer Chain Length.

| Kinematic | | | Specific | Surface | Refractive |
|---|---|---|---|---|---|
| Viscosity, | Flashpoint, ° C. | Freezing | Gravity, | Tension, | Index, |
| cSt | COC | Point, ° C. | at 25° C. | mN/m | at 25° C. |
| 0.65 | −4 | −67 | 0.760 | 15.9 | 1,375 |
| 1 | 40 | −85 | 0.816 | 17.4 | 1,382 |
| 2 | 48 | −90 | 0.830 | 18.1 | 1,387 |
| 3 | 62 | −100 | 0.900 | 18.9 | 1,392 |
| 5 | 136 | −100 | 0.910 | 19.7 | 1,397 |
| 10 | 162 | −65 | 0.930 | 20.1 | 1,399 |
| 20 | 230 | −60 | 0.950 | 20.6 | 1,400 |
| 50 | 280 | −55 | 0.959 | 20.7 | 1,402 |
| 100 | >300 | −55 | 0.965 | 20.9 | 1,403 |
| 200 | >300 | −50 | 0.970 | 21.0 | 1,403 |
| 300 | >300 | −50 | 0.970 | 21.1 | 1,403 |
| 350 | >300 | −50 | 0.970 | 21.1 | 1,403 |
| 500 | >300 | −50 | 0.970 | 21.1 | 1,403 |
| 1000 | >300 | −50 | 0.970 | 21.2 | 1,403 |
| 5000 | >300 | −50 | 0.975 | 21.4 | 1,403 |
| 10000 | >300 | −50 | 0.975 | 21.5 | 1,403 |
| 12500 | >300 | −50 | 0.975 | 21.5 | 1,403 |
| 30000 | >300 | −50 | 0.975 | 21.5 | 1,403 |
| 60000 | >320 | −50 | 0.975 | 21.5 | 1,403 |
| 100000 | >300 | −50 | 0.976 | 21.5 | 1,404 |
| 300000 | >300 | −45 | 0.976 | 21.5 | 1,404 |
| 1000000 * | >300 | −40 | 0.976 | 21.5 | 1,404 |

* 1000000 cSt, longest polymer chain.

Silicone oils having a dynamic viscosity in the range of approximately 0.65 to approximately 10.0 mPa·s are suitable in certain embodiments. However, silicone oils having a dynamic viscosity in the range of approximately 1.0 to approximately 3.0 mPa·s may be particularly suited for certain embodiments. Both volatility and viscosity of the silicone oils according to at least some embodiments are obtained in that range, so that such silicone oils can be easily infused into the posterior chamber through current surgical cannulas. In certain embodiments, the silicone oils comprise a volatility of sufficient magnitude so that it can exit the eye cavity by way of evaporating through other surgical incisions. In certain embodiments, the silicone oil is absorbed by capillaries and removed at least partially due to the concentration gradient after an intended use.

Table 2 compares various properties of oil solvents with those of other oil solvents. Table 3 shows the physical/chemical properties of Silicone oils versus that of various rinsing agents and oil solvents. Table 4 shows the physical/chemical properties of three surfactants.

TABLE 2
Relative Properties of Oil Solvents

| | | Saline | F6H8 | PFOB | F4H5 |
|---|---|---|---|---|---|
| Status | R&D | Clinical | Clinical | Clinical | R&D, |
| | | | | | Clinical Data |
| Availability | Commercial | Commercial | Only one | Commercial | Only one |
| | | | provider | | provider |
| Efficacy | 1* | 5 | 3 | 3 | 2 |
| Volatility | 1* | 5 | 3 | 4 | 2 |
| Cell toxicity | 3 | 4 | 2 | 1 | N/A |
| Cell proliferation | 2 | 1 | 2 | 3 | N/A |
| Manufacturing | Low | Cheapest | Medium-high | Medium | High |
| cost | | | | | |
| Kinematic | 0.65/ | 0.89 | 3.44 | 1.93 | 1.05 |
| Viscosity | 1.0 cSt | | | | |
| (Sticky**) | | | | | |

*1 = most efficient/toxic/proliferative/volatile
**Since the viscosity of one or more embodiments of the present application is similar to water, it is easy to be aspirated through air/fluid exchange during surgical procedure.

TABLE 3
Physical/Chemical Properties of Silicone Oils, Rinsing Agents, and Oil Solvents

| | | | | Dynamic | | | |
|---|---|---|---|---|---|---|---|
| | Molecular | Density | Boiling | Viscosity | Vapor | Surface | Interface |
| | Weight | (g/cm³) | Point | (mPa · s) | Pressure | Tension | Tension |
| Substance | (g/mol) | at 25° C. | (° C.) | at 25° C. | (Torr) | (mN/m) | (mN/m) |
| F4H5 | 290 | 1.284 | 134 | 1.05 | 25.1 (37° C.) | 17.43 | 43.0 |
| F6H6 | 404 | 1.386 | 187 | 2.38 | <1 (25° C.) | 20.0 | 49.6 |
| F6H8 | 432 | 1.331 | 223 | 3.44 | <1 (25° C.) | 19.65 | 45.3 |
| F6H12 | 488 | 1.25 | 290 | 6.99 | — | 21.1 | — |
| PFOB | 499 | 1.930 | 143 | 1.93 | 10.5 (37° C.) | 18 | 51.3 |
| Hexamethyl- | 162.38 | 0.764 | 99 | 0.65 | 42.2 (37° C.) | 15.9 | — |
| disiloxane | | (20° C.) | | | | | |
| Octamethyl- | 236.53 | 0.818 | 153 | 1.0 | 4 (25° C.) | 17.4 | — |
| trisiloxane | | | | | | | |
| H₂O | 18 | 0.970 | 100 | 0.89 | 46.9 (37° C.) | 72 | — |
| Silicone Oil | — | 0.97 | — | 1000 | — | 20.9 | 39.4 |
| 1000 | | | | | | | |
| Silicone Oil | — | 0.97 | — | 5000 | — | 20.8 | 43.0 |
| 5000 | | | | | | | |

TABLE 4
Physical/Chemical Properties of Surfactants

| | Octamethylcyclo- | Decamethylcyclo- | Dodecamethylcyclo- |
|---|---|---|---|
| | tetrasiloxane | pentasiloxane | bexasiloxane |
| Melting Point, ° C. | 17.7 | -38 | -3 |
| Boiling Point, ° C. | 175 | 211 | 245 |
| Density, g/cm³ at 25 ° C. | 0.95 | 0.954 | 0.963 |
| Vapor Pressure, Pa at 25° C. | 132 | 33.2 | 4.6 |
| Water Solubility, mg/L at 23° C. | 0.056 | 0.017 | 0.053 |
| Henry's Law Constant, | 1,214,000 | 3,342,000 | 14,667 |
| Pa m³/mol at 25° C. | | | |
| Heat of Evaporation, kJ/mol | 44 | 51.4 | — |

In certain embodiments, the silicone oils that are suitable for the rinsing agent comprise hexamethyldisiloxane, octamethyltrisiloxane, decamethyltetrasiloxane, dodecamethylpentasiloxane, dimethicone or a combination thereof. It should be noted that the aforementioned list of silicone oils is not an exhaustive list.

A presence of a hydrophobic surfactant in the rinsing agent may serve a useful purpose, at least in particular embodiments. As discussed heretofore, a hydrophobic surfactant promotes formation of double emulsion, which stabilizes aqueous globules that encapsulate emulsified oil droplets within the rinsing agent. In one or more embodiments, the hydrophobic surfactant included in the rinsing agent is oil soluble.

A relevant property of a hydrophobic surfactant according to certain embodiments is a property of the relative hydrophobicity and amphiphilicity. In accordance with the Bancroft Rule, “The phase in which a surfactant is more soluble constitutes the continuous phase.” A hydrophobic nature of a surfactant facilitates, at least in part, formation of water-in-oil (w/o) emulsion. A medical problem that the rinsing agent is designed to address is the removal of an o/w emulsion in the eye cavity. Thus, a hydrophobic property triggers and/or promotes formation of oil-in-water-in-oil (o/w/o) double emulsion. One index used for evaluating the hydrophobicity of a substance is its Hydrophilic-Lipophilic Balance (HLB) number. In certain embodiments, the surfactant has an HLB number range from 6 to 7, 7 to 8, 8 to 9, 9 to 10, 10 to 11, and 11 to 12.

In certain embodiments, a second relevant property of a hydrophobic surfactant is solubility in low-MW silicone oil. Surfactants that are soluble in certain silicone oils induce formation of the double emulsion within the rinsing agent.

An example of the surfactant that is suitable for serving as the active ingredient in one or more embodiments is Dow Corning DC749 fluid, which includes a blend of approximately 50.0% trimethylsiloxysilicate and approximately 50.0% cyclomethicone (decamethylpenta-cyclosiloxane). Dow Corning DC749 fluid is a silicone-based surfactant and therefore it is highly soluble in low-molecular-weight silicone oil and it is hydrophobic in nature. The material can be purchased from Dow Corning Corporation, Corporate Center, PO Box 994, MIDLAND MI 48686-0994, USA. In other embodiments, a blend of 10-20% trimethylsiloxysilicate and 80-90% cycolmethicone; 20-30% trimethylsiloxysilicate and 70-80% cycolmethicone; 30-40% trimethylsiloxysilicate and 70-80% cycolmethicone; 40-50% trimethylsiloxysilicate and 60-70% cycolmethicone; 80-90% trimethylsiloxysilicate and 10-20% cycolmethicone; 70-80% trimethylsiloxysilicate and 20-30% cycolmethicone; 70-80% trimethylsiloxysilicate and 20-30% cycolmethicone; 80-90% trimethylsiloxysilicate and 10-20% cycolmethicone. It should be noted that the aforementioned material is intended only to provide an example of a suitable hydrophobic surfactant for use in a rinsing agent.

In at least one embodiment, the v/v ratio of the hydrophobic surfactant in the rinsing agent ranges from 0% to 1% v/v, 1% to 2% v/v, 2% to 3% v/v, 3% to 4% v/v, 4% to 5% v/v, 5% to 6% v/v, 6% to 7% v/v, 7% to 8% v/v, 8% to 9% v/v, 9% to 10% v/v, 10% to 11% v/v, 11% to 12% v/v, 12% to 13% v/v, 13% to 14% v/v, 14% to 15% v/v, 15% to 16% v/v, 16% to 17% v/v, 17% to 18% v/v, 18% to 19% v/v, and 19% to 20% v/v.

In certain embodiments, the rinsing agent may also consist of one or more additional therapeutic agents. The additional therapeutic agents include, but are not limited to, proteins and proteinaceous compounds, steroids (including angiostatic or anti-inflammatory steroids), nonsteroidal anti-inflammatories, antibacterials, anti-infective agents, antivirals, antifungals, insulins, Glugacon-like Peptide-1 (GLP-1) and its analogs, Beta Adrenergic Blocking Agents (Beta blockers), antihistamines, anti-microtubule agents, vitrectomy agents, therapeutic antibodies, anti-glaucoma agents, buffering agents, tonicity agents, viscosity enhancing agents, preservatives, and healing agents (e.g., vitamins).

Proteins and proteinaceous compounds that can be included in the rinsing agent include, but are not limited to, antibodies, growth hormone, Factor VIII, Factor IX and other coagulation factors, chymotrypsin, trysinogen, alpha-interferon, beta-galactosidase, lactate dehydrogenase, growth factors, clotting factors, enzymes, immune response stimulators, cytokines, lymphokines, interferons, immunoglobulins, retroviruses, interleukins, peptides, somatostatin, somatotropin analogues, somatomedin-C, Gonadotropic releasing hormone, follicle stimulating hormone, luteinizing hormone, LHRH, LHRH analogues such as leuprolide, nafarelin and geserelin, LHRH agonists and antagonists, growth hormone releasing factor, callcitonin, colchicines, gonadotropins such as chorionic gonadotropin, oxytocin, octreotide, somatotropin plus and amino acid, vasopressin, adrenocorticotrophic hormone, epidermal growth factor, prolactin, somatotropin plus a protein, cosyntropin, lypressin, polypeptides such as thyrotropin releasing hormone, thyroid stimulation hormone, secretin, pancreozymin, enkephalin, glucagons, and endocrine agents.

Angiostatic and/or anti-inflammatory steroids that can be included in one or more embodiments include, but are not limited to anecortive acetate (Retaane®, Alcon, Inc., Fort Worth, Tex.); tetrahydrocortisol; 4,9(11)-pregnadien-17α,21-diol-3,20-di-one (Anecortave) and its -21-acetate salt; 11-epicortisol; 17α-hydroxyprogesterone; tetrahydrocortexolone; cortisona; cortisone acetate; hydrocortisone; hydrocortisone acetate; fludrocortisone; fludrocortisone acetate; fludrocortisone phosphate; prednisone; prednisolone; prednisolone sodium phosphate; methylprednisolone; methylprednisolone acetate; methylprednisolone, sodium succinate; triamcinolone; triamcinolone-16,21-diacetate; triamcinolone acetonide and its -21-acetate, -21-disodium phosphate, and -21-hemisuccinate forms.

Nonsteroidal anti-inflammatories that can be included in the rinsing agent include, but are not limited to naproxin; diclofenac; celecoxib (Celebrex®, Pfizer); sulindac; diflunisal; piroxicam; indomethacin; etodolac; meloxicam; ibuprofen; ketoprofen; r-flurbiprofen (Myriad Genetics, Inc.); mefenamic; nabumetone; tolmetin, and sodium salts of each of the foregoing; ketorolac tromethamine; ketorolac tromethamine (Acular®, Allergan, Inc.); choline magnesium trisalicylate; rofecoxib; valdecoxib; lumiracoxib; etoricoxib; aspirin; salicylic acid and its sodium salt; salicylate esters of α,β,γ-tocopherols and tocotrienols (and all their d, I, and racemic isomers); methyl, ethyl, propyl, isopropyl, n-butyl, sec-butyl, t-butyl, esters of acetylsalicylic acid; tenoxicam; aceclofenac; nimesulide; nepafenac; amfenac; bromfenac; flufenamate; and phenylbutazone.

Antibacterials that can be included in the rinsing agent include, but are not limited to aztreonam; cefotetan and its disodium salt; loracarbef; cefoxitin and its sodium salt; cefazolin and its sodium salt; cefaclor; ceftibuten and its sodium salt; ceftizoxime; ceftizoxime sodium salt; cefoperazone and its sodium salt; cefuroxime and its sodium salt; cefuroxime axetil; cefprozil; ceftazidime; cefotaxime and its sodium salt; cefadroxil; ceftazidime and its sodium salt; cephalexin; cefamandole nafate; cefepime and its hydrochloride, sulfate, and phosphate salt; cefdinir and its sodium salt; ceftriaxone and its sodium salt; cefixime and its sodium salt; cefpodoxime proxetil; meropenem and its sodium salt; imipenem and its sodium salt; cilastatin and its sodium salt; azithromycin; clarithromycin; dirithromycin; erythromycin and hydrochloride, sulfate, or phosphate salts ethylsuccinate, and stearate forms thereof, clindamycin; clindamycin hydrochloride; sulfate or phosphate salt; lincomycin and hydrochloride, sulfate, or phosphate salt thereof; tobramycin and its hydrochloride, sulfate, or phosphate salt; streptomycin and its hydrochloride, sulfate, or phosphate salt; vancomycin and its hydrochloride, sulfate, or phosphate salt; neomycin and its hydrochloride, sulfate, or phosphate salt; acetyl sulfisoxazole; colistimethate and its sodium salt; quinupristin; dalfopristin; amoxicillin; ampicillin and its sodium salt; clavulanic acid and its sodium or potassium salt penicillin G; penicillin G benzathine, or procaine salt; and penicillin G sodium or potassium salt.

Anti-infective agents that can be included in the rinsing agent include, but are not limited to 2,4-diaminopyrimidines (e.g., brodimoprim, tetroxoprim, trimethoprim); nitrofurans, (e.g., furaltadone, furazolium chloride, nifuradene, nifuratel, nifurfoline, nifurpirinol, nifurprazine, nifurtoinol, nitrofuirantoin); quinolones and analogs (e.g., cinoxacin, ciprofloxacin, clinafloxacin, difloxacin, enoxacin, fleroxacin, flumequine, gatifloxacin, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin); sulfonamides (e.g., acetyl sulfamethoxypyrazine, benzylsulfamide, chloramine-b, chloramine-t, dichloramine t, n²-formylsulfisomidine, n⁴-β-d-glucosylsulfanilamide, mafenide, 4′-(methylsulfamoyl) sulfanilanilide, noprylsulfamide, phthalylsulfacetaride, phthalylsulfathiazole, and salazosulfadimidine.

Antivirals that can be included in the rinsing agent include, but are not limited to amprenavir; interferon alfa-n3; interferon alfa-2b; interferon alfacon-1; peginterferon alfa-2b; interferon alfa-2a; lamivudine; zidovudine; amadine (Symmetrel®, Endo Pharm, Inc.) and its hydrochloride, sulfate, and phosphate salts, indinavir and its hydrochloride, sulfate, or phosphate salt; ganciclovir; ganciclovir sodium salt; famciclovir; rimantadine and its hydrochloride, sulfate, or phosphate salt; saquinavir mesylate; foscamet; zalcitabine; ritonavir; ribavirin; zanamivir; delavirdine mesylate; efavirenz; amantadine and its hydrochloride, sulfate, or phosphate salt; palivizumab; and oseltamivir and its hydrochloride, sulfate or phosphate salt.

Antifungals that can be included in the rinsing agent include, but are not limited to amorolfine, amphotericin B, anidulafungin, butoconazole, butenafine, caspofungin, ciclopirox olamine, clotrimazole, econazole, fluconazole, flucytosine, griseofulvin, haloprogin, itraconazole, ketoconazole, micafungin, miconazole (including miconazole nitrate), naftifine, nikkomycin Z, nystatin (topical and liposomal), oxiconazole, posaconazole, pimaricin, ravuconazole, sulconazole, terbinafine, terconazole, tioconazole, tolnaftate, undecylenate, and voriconazole.

Insulins that can be included in the rinsing agent include, but are not limited to Novolog® (insulin aspart [rDNA origin]) and Novolin® products (Novo Nordisk Inc.); Humalog® (insulin lispro [rDNA origin]), Humalog® 75/25 and 50/50 (mixtures of insulin lispro protamine suspension and insulin lispro), and Humalin® products (regular human insulin [rDNA origin], Eli Lilly & Co.); Lantus® (insulin glargine [rDNA origin], Sanofi Aventis U.S. LLC); and porcine and bovine insulins.

GLP-1 and analogs (for diabetes therapy, appetite suppression, cardiac protection) (see Keiffer et al., 20 Endocr Rev., 876-913 (1999)) that can be included in the rinsing agent include, but are not limited to liraglutide (Novo Nordisk Inc.); GLP-1 receptor simulators such as Byetta® products (exenatide, and incretin mimetic, Amylin Pharm., Inc./Eli Lilly & Co.) and ZP-10 (Zealand Pharma A/S), GLP-1-albumin (ConjuChem Inc.); DPP-IV inhibitors (which inhibit enzyme attack on GLP-1) such as Galvus® (vildagliptin, formerly LAF237, Novartis), Januvia® (sitagliptin, formerly MK-0431, Merck & Co.); saxagliptin (formerly BMS-477188, Bristol-Myers Squibb), and GSK23A (GlaxoSrnithKline).

Beta blockers that can be included in the rinsing agent include, but are not limited to betaxolol (Betoptic®, Betoptic® S betaxolol hydrochloride suspension, Alcon Labs., Inc.), and its hydrochloride, sulfate, or phosphate salt; levobetaxolol and its hydrochloride, sulfate, or phosphate salt; and timolol maleate (Timoptic®, Timoptic-XE®, timolol maleate ophthalmic gel-forming solution, Merck & Co.), levobunolol (Betagan®, levobunolol hydrochloride ophthalmic solution, Allergan), carteolol (Ocupress®, carteolol hydrochloride ophthalmic solution, CIBA Vision Sterile Mfg./Novartis Ophthalmics), and metipranolol (OptiPranolol®, metipranolol ophthalmic solution, Bausch & Lomb).

Antihistamines that can be included in the rinsing agent include, but are not limited to olopatadine and its hydrochloride, sulfate, or phosphate salt forms; fexofenadine and its hydrochloride, sulfate, or phosphate salt; azelastine and its hydrochloride, sulfate, or phosphate forms; diphenhydramine and its hydrochloride, sulfate, or phosphate forms; and promethazine and its hydrochloride, sulfate, or phosphate forms.

Antimicrotubule agents that can be included in the rinsing agent include, but are not limited to Taxoids including paclitaxel (Taxol®, Bristol-Myers Squibb); vincristine (Oncovin®, Eli Lilly & Co.) and its hydrochloride, sulfate, or phosphate salt forms; vinblastine (Velbe®, Eli Lilly & Co.), and its hydrochloride, sulfate, or phosphate salt; vinorelbine (Navelbine®, Fabre Pharm. Inc.); colchicines; docetaxel (Taxotere®, Sanofi-Aventis U.S. LLC); RPR-109881 (Sanofi-Aventis); LIT 976 (Sanofi-Aventis); BMS 188797 and BMS 184476 (Bristol-Myers Squibb); DJ 927 (Daiichi Pharm. Inc.); DHA-paclitaxel (Taxoprexin®, Protarga, Inc.); Epothilones including epothiloneB such as patupilone (EPO 906, Novartis/generic), BMS 247550 and BMS-310705 (Bristol-Myers Squibb), epothilone D (KOS 862, Kosan Biosci. Inc.) and ZK EPO (Schering AG).

Vitrectomy agents, such as hyaluronidase (Vitrase®, ISTA Pharm., Inc.) can also be included in the rinsing agent in one or more embodiments.

Therapeutic antibodies that can be included in the rinsing agent include, but are not limited to Herceptin® (trastuzumab, Genentech. Inc.). MDX-H210 (Medarex, Inc.); SGN-15 (Seattle Genetics); H11 (Viventia); Therex (Antisoma); rituximan (Rituxan®, Genentech); Campath (ILEX Oncology/Millennium/Shering); Mylotarg (Celltech/Wyeth); Zevalin (IDEC Pharmaceuticals/Schering); tositumomab; (Bexxar®, GlaxoSmithKline); epratuzumab (Lymphocide, Immunomedics/Amgen); Oncolym® (Techniclone Corp./Schering AG); Mab Hu1D10 antibody (Protein Design Laboratories); ABX-EGF (Abigenix); infleximab (Remicade®, Centocor) and etanercept (Enbrel®, Wyeth-Ayerst).

Anti-glaucoma agents that can be included in the rinsing agent include, but are not limited to prostaglandins: latanoprost, bimaloprost, travoprost; dorzolamide (Cosopt™ dorzolamide hydrochloride hydrochloride-timolol maleate ophthalmic solution, Merck); blockers: timolol (acid-free and amine salts forms), levobunolol, betaxolol (Kerlone® beta-adrenergic blocking agent, Sanofi-Aventis), and its hydrochloride, sulfate, phosphate salts; atenolol; a 2-adrenergic antagonists: brimonidine; sympathmimetics: epinephrine, dipivetrin; miotic agents: philicarpine; carbonic anhydrase inhibitors; dorzolamide, brinzolamide, acetolamide; and chlorthalidone (PLIVA®, Inc., East Hanover, N.J.).

A buffering agent may be used to maintain the pH of any ophthalmologic compositions of the present application, for example, eye drop formulations, in the range of about 4.0 to about 8,0; so as to minimize potential irritation to the eye. In certain embodiments, the pH is maintained at about 3.5 to about 6.0, preferably about 4.0 to about 5.5, in order to ensure that most of the hydroxylamine is in its protonated form for highest aqueous solubility. The buffer may be any weak acid and its conjugate base with a pKa of about 4.0 to about 5.5; e.g., acetic acid/sodium acetate; citric acid/sodium citrate. The pKa of the hydroxylamines is about 6.0. For direct intravitreal or intraocular injection, formulations should be at pH 7.2 to 7.5, preferably at pH 7.3-7.4.

The ophthalmologic compositions may also include tonicity agents suitable for administration to the eye. Among those suitable is sodium chloride to make formulations of the present invention approximately isotonic with 0.9% saline solution.

In one or more embodiments, the compositions are formulated with viscosity enhancing agents. Exemplary agents are hydroxyethylcellulose, hydroxypropylcellulose, methylcellulose, and polyvinylpyrrolidone. The viscosity agents may be present in the compounds up to about 2.0% weight by volume. It may be preferred that the agents are present in a range from about 0.2% to about 0.5% weight by volume. A preferred range for polyvinylpyrrolidone may be from about 0.1% to about 2.0% weight by volume. One skilled in the art may prefer any range established as acceptable by the Food and Drug Administration.

Preservatives may be used in one or more embodiments within particular ranges. Among those preferred are up to 0.013% weight by volume of benzalkonium chloride, up to 0.013% weight by volume of benzethonium chloride, up to 0.5% weight by volume of chlorobutanol, up to 0.004% weight by volume or phenylmercuric acetate or nitrate, up to 0.01% weight by volume of thimerosal, and from about 0.01% to about 0.2% weight by volume of methyl or propylparabens.

In one or more embodiments, the composition may include a healing agent. Healing agents that can be included in the rinsing agent include, but are not limited to vitamin A, vitamin D, vitamin E and vitamin K, alpha-tocopherol derivatives, retinol derivatives, lutein, aloe vera extracts such as aloine, omega-3 fatty acids, cyanocobalamin, L-cystine, pyridoxine, acetylcysteine, essential oils such as oil of calendula, cedar, lavender and their analogs and derivatives.

Those of ordinary skill in the art will appreciate that any of the foregoing disclosed active agents may be used in combination or mixture in the pharmaceutical formulations of the present application. Such mixtures or combinations may be delivered in a single formulation, or may be embodied as different formulations delivered either simultaneously or at distinct time points to affect the desired therapeutic outcome.

The formulations of the present application may be sterilized for use by methods known to those of ordinary skill in the art.

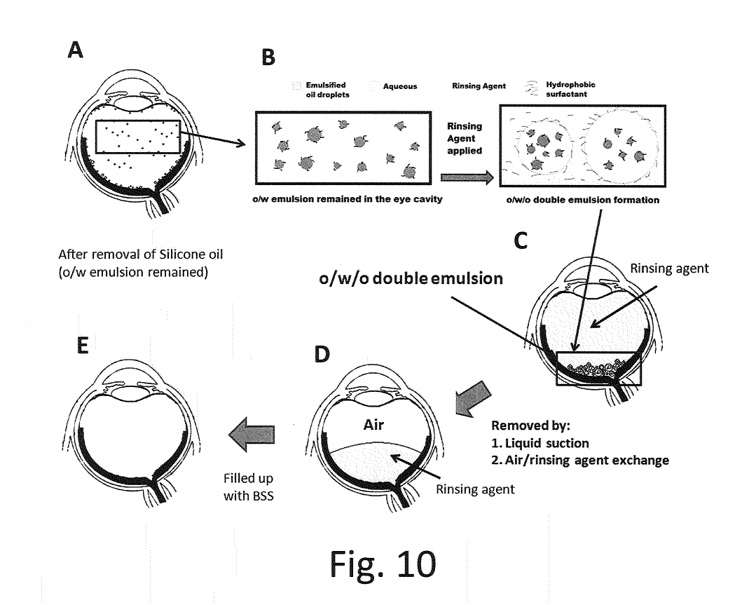

FIGS. 10A-E are diagrams illustrating a method of using an ophthalmological rinsing agent according to an embodiment. FIG. 10(A) shows, after the removal of the bulk silicone oil, emulsified silicone oil droplets (in the form of o/w emulsion) may remain. FIG. 10(B) shows mixing a relatively large amount of the rinsing agent with the o/w emulsion in the eye cavity triggers the formation of the o/w/o double emulsion due to the presence of hydrophobic surfactant in the rinsing agent. Unwanted emulsified silicone oil droplets are being encapsulated by the larger aqueous globules. FIG. 10(C) shows aqueous globules encapsulated with emulsified oil droplets gathering at a bottom portion of the eye cavity as a result of a higher density of aqueous solution than rinsing agent. FIG. 10(D) shows that doubly emulsified droplets may be removed by suction and by repeatedly permitting an air/rinsing agent exchange. FIG. 10(E) shows surgical long acting gas, such as SF₆, may be used to fill up the eye cavity at the end of the operation after the rinsing agent has been removed.

The following examples refer to the efficacy and safety of rinsing agents in accordance with one or more embodiments of the present application.

### 6.1. Efficacy of Encapsulation of Emulsified Oil Droplets

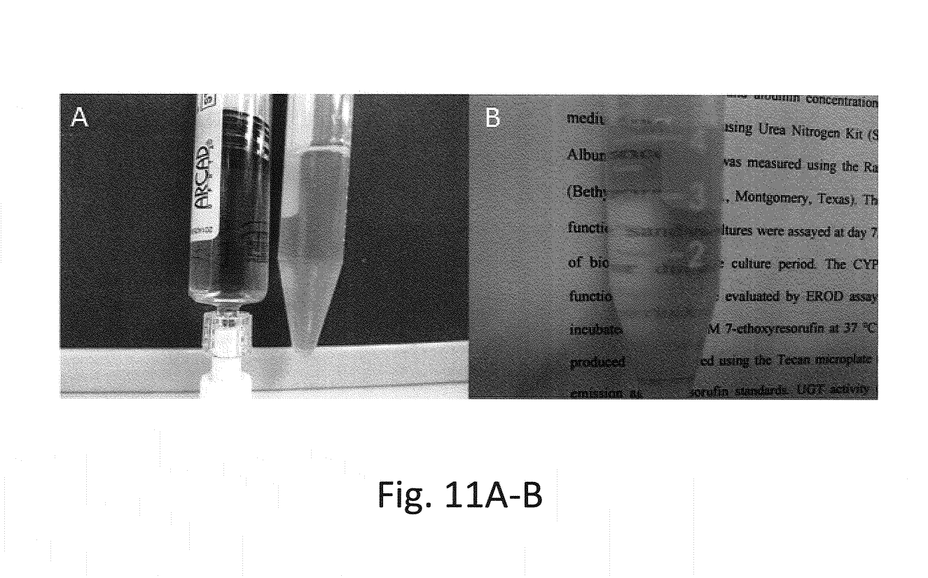

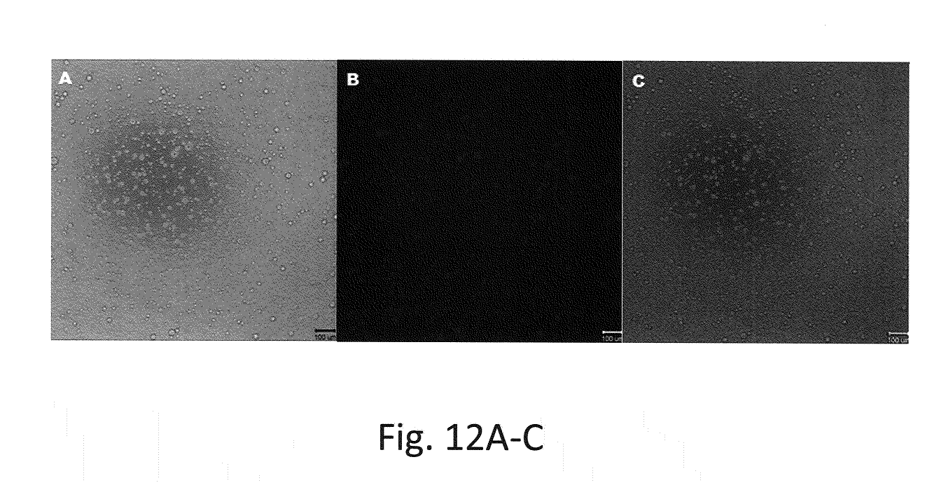

Experiments were done to prove the efficacy of the encapsulation of the emulsified oil droplets by a rinsing agent in accordance with at least one embodiment. 1300 cSt silicone oil was labeled by a green fluorescent dye BODIPY (493/503) (FIG. 11A-B). Standard emulsion was then generated by homogenizing the fluorescent labeled 1300 cSt silicone oil together with 2% Pluronic F68 (hydrophilic surfactant) in 1× Phosphate Buffered Saline (PBS) in a volume ratio of oil to the aqueous solution of 1:99 by a high-speed disperser (T-10 Basic, IKA) at a speed of 30000 rpm for 1 minute (FIG. 12A-C). This silicone-oil-in-water emulsion was then mixed with the proposed rinsing agent in a small chamber.

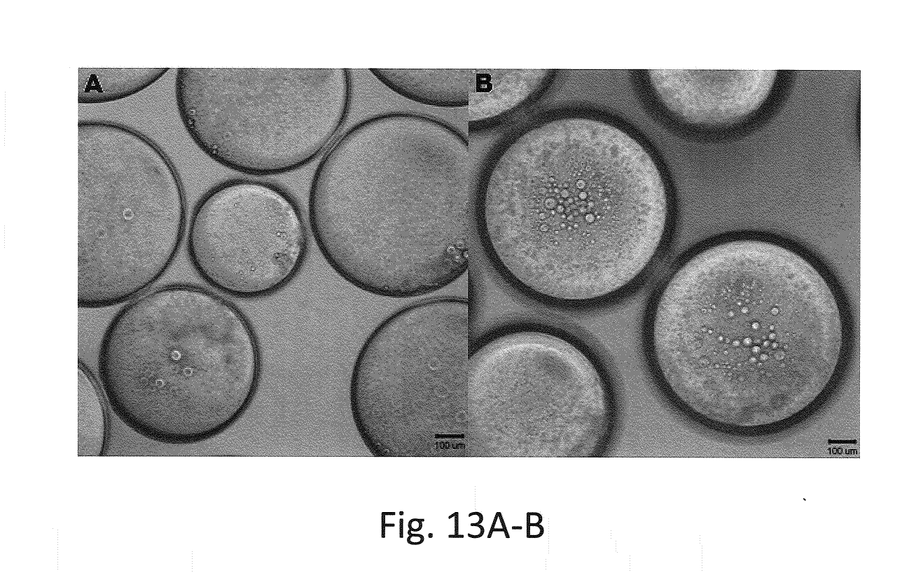

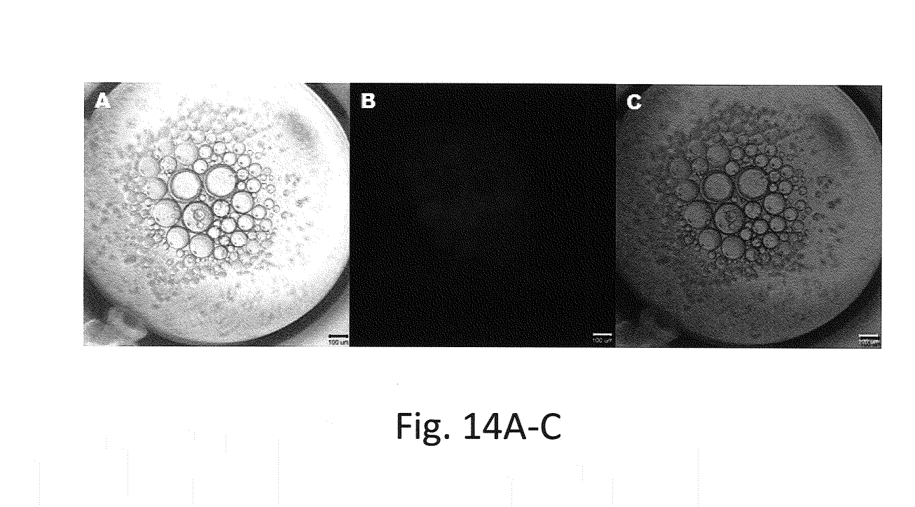

Microscopic examination was done to visualize the formation of oil-in-water-in-oil double emulsion. After washing with the proposed rinsing agent, three phases with two distinct boundaries were observed indicating the formation of double emulsions, as shown in FIG. 13. In FIG. 14(C), the merged image further supported our predicted formation of o/w/o double emulsion as the innermost phase showed a green fluorescence signal after the innermost silicone oil droplets are dyed with an oil-soluble fluorescence dye.

### 6.2 Efficiency of the Rinsing Agent in Removing the Emulsified Oil Droplets in an In Vitro Washing Eye Model Chamber

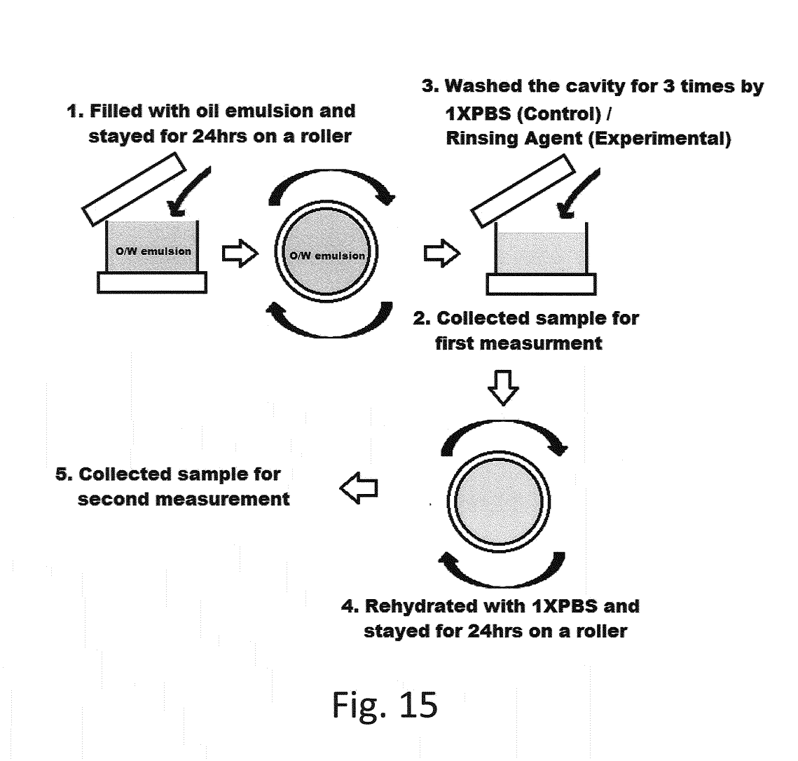

In this example, a custom-built polymethylmethacrylate (PMMA) model eye chamber served as an in-vitro eye model. Standard emulsion was kept in the in-vitro eye model for 24 hours. After that, emulsion inside the chamber was collected and the droplet size was measured using the Coulter counter Multisizer 4 to obtain the “original” count of the emulsion droplets inside the chamber. The chambers were subsequently rinsed by saline for 4 times (as control), and, by the proposed rinsing agent 3 times followed by saline for a single time (as the experimental run). After the rinsing steps, the chambers were rehydrated with 1×PBS. The rinsed chambers were then subjected to continuous rolling motion, which simulated the motion of the eyeball, for another 24 hours. After that, the liquid inside the chamber was collected for a second measurement of the sizes of any droplets left using the Coulter Counter Multisizer 4 (FIG. 15). Successful removal of the emulsified oil will lead to a significantly reduced number of droplets remained in the model eye chamber.

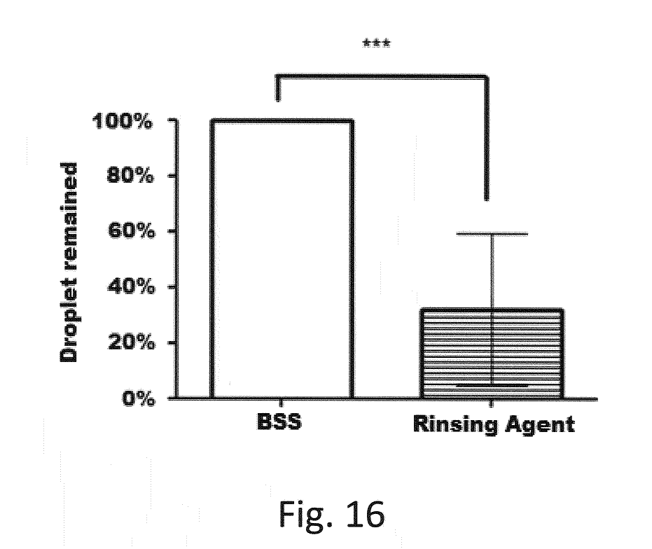

The result showed that the amount of emulsified droplets remained in the chamber after washing by the proposed rinsing agent was only one-third of the droplets of that after washing by the saline (FIG. 16), indicating its higher efficiency in removing the emulsified droplets in this application. In summary, the rinsing agent effectively removes emulsified silicone oil droplets in vitro. Since the components are widely used in many fields, especially in cosmetics and medicine, it is predicted to have less safety concern and issues during registration and clinical practice.

### 6.3 Encapsulation of Emulsified Oil Droplets and Efficiency of the Rinsing Agent in Removing the Emulsified Oil Droplets

In this study, the rinsing agent consisted of hexamethyldisiloxane, the monomer of polydimethylsiloxanc (PDMS) as the major component with a small portion of a silicone-based hydrophobic surfactant Dow Corning 749 fluid. Silicone oil 1300 cSt was stained with BODIPY 493/503 (Invitrogen) and then dispersed in 4% Pluronic F68 (Sigma) in 1× phosphate buffer saline (PBS) to form an oil-in-water (o/w) emulsion. This emulsion was then added to and kept in an eye model chamber for 24 hours. The chamber was subsequently washed with the proposed rinsing agent or 1×PBS in the control experiment. The washout was collected and observed under an optical microscope. The chamber after rinsing was then filled with 1×PBS. After 24 hours, the number of emulsified oil droplets was quantified using a particle counter Coulter counter Multisizer 4.

The results showed that, in the in-vitro washing model, rinsing by the proposed washing agent led to a reduction of oil droplets; the amount of oil droplets remaining is only one-third of that after washing by 1×PBS. Moreover, an oil-in-water-in-oil (o/w/o) double emulsion was observed in the washout of the proposed rinsing agents. Fluorescence signals due to BODIPY were detected within the larger water droplets; this confirms that the initial SO droplets were encapsulated by an aqueous shell phase. The resultant o/w/o double emulsion could be washed away easily, while the remaining washing agent can be easily removed by evaporation.

This study showed that emulsified SO droplets can be encapsulated within water droplets with the use of the proposed rinsing agent and the resultant o/w/o double emulsion can be easily washed away. Therefore, this rinsing agent, in accordance with at least one embodiment of the present application, demonstrates an excellent potential in reducing the postoperative complications associated with emulsification after the removal of silicone oil.

### 6.4 Cell Viability and Cell Death of the RGC-5 and rMC-1 Cells after Incubation with Rinsing Agent for 1-Hr In Vitro

The innermost cell layer of the retina is called the retinal ganglion cell layer. It consists mainly of retinal ganglion cells, with some Müller cells and also some blood capillaries. This cell layer is located closest to the vitreous anatomically and thus is almost in direct contact with the rinsing agent in this study. Therefore, a retinal ganglion cell line (RGC-5) and a rat Müller cell line were used in this in-vitro cell culture study to evaluate the biocompatibility of the rinsing agent in accordance with at least one embodiment of this application.

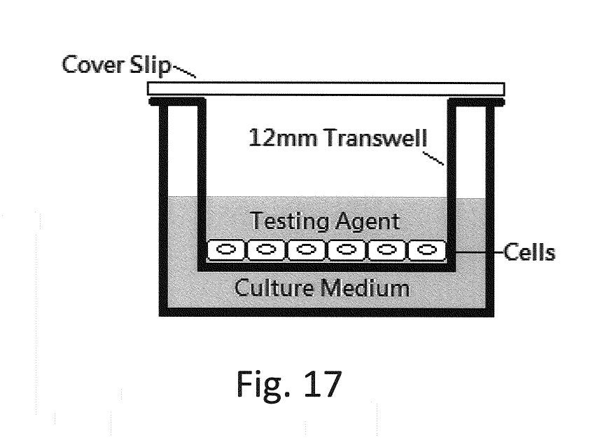

In the study, CellTiter 96® AQᵤₑₒᵤₛ non-radioactive cell proliferation assay (MTS) and Cytotoxicity Detection Kit (LDH assay) were used to test for the cell viability and cell death of the RGC-5 and rMC-1 cells after incubation with the rinsing agent (FIG. 17). The major component of the rinsing agent is octamethyltrisiloxane (1.0 cSt Silicone Oil [SO]) with hydrophobic surfactant DC749 (Dow Corning). Rinsing agents with different concentration of DC749 (0%, 5%, 10% in vol.) were included. The incubation time was designed as 1 hr because the proposed use of the rinsing agent would not be longer than 15 mins in-vivo and therefore a 1 hr study would be more than sufficient in deciding the effect of the proposed rinsing agent on the cells in the eye cavity. Hank's Balanced salt solution (HBSS) was used as the experimental control. Two liquids, perfluorodecalin (PFD) and F6H8, were also included in this study. PFD is a widely accepted short-term used intraoperative adjunct for flattening the folded retina during retinal surgery. F6H8 is a key component of Heavy Silicone Oil (HSO), which has been used as a long-term intraocular tamponade. The performance of the rinsing agent groups was compared with PFD and F6H8 which have been treated as the acceptable norm in clinical use.

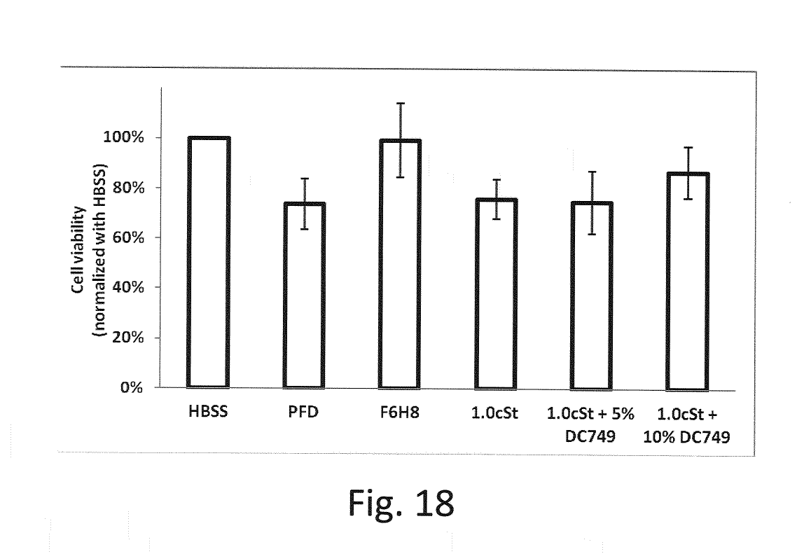

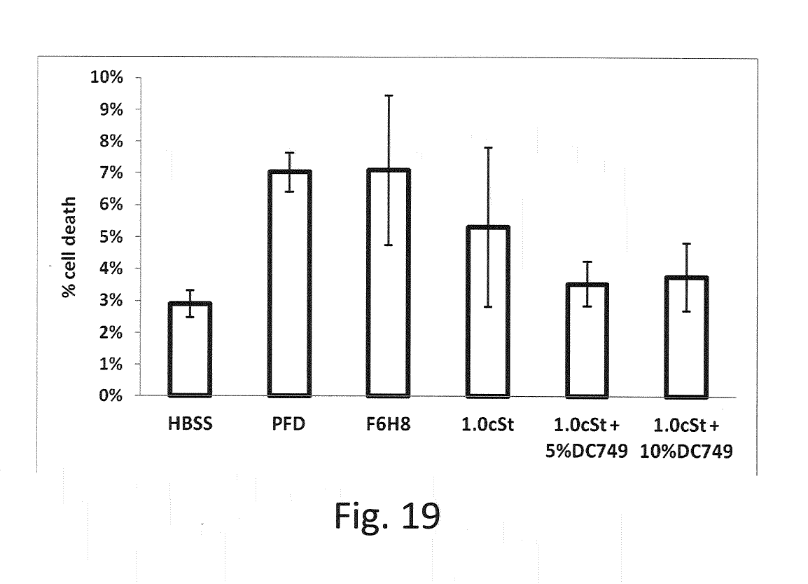

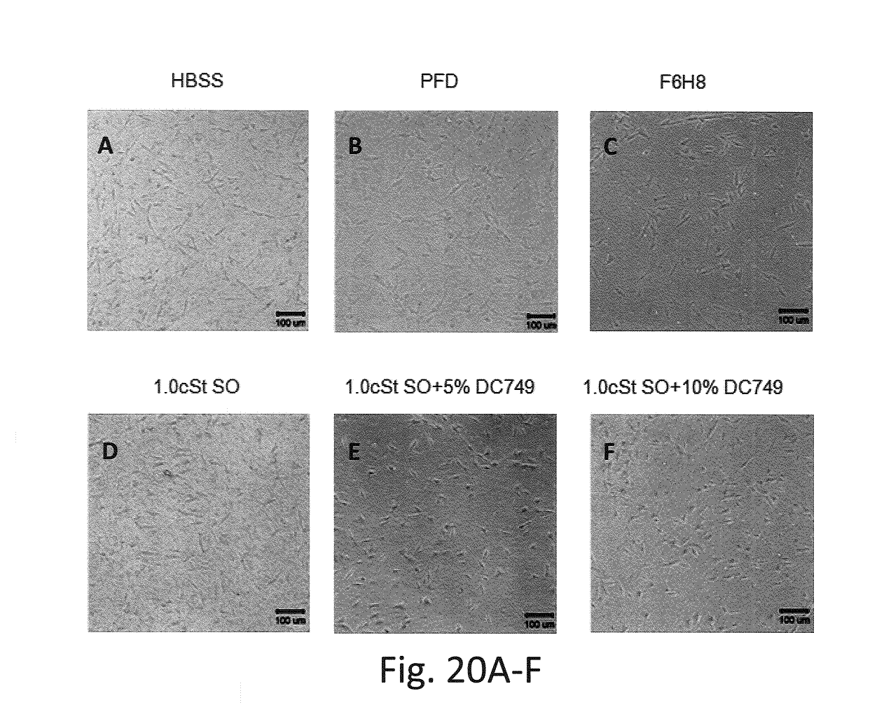

Experiments were performed three times for both the MTS and LDH assays. In RGC-5 study, no difference in both the relative cell viability and percentage of dead cell could be observed in all the experimental groups using the proposed rinsing agents with different concentrations of surfactant, when compared with the control, HBSS, and also the references, PFD and F6H8, by one-way ANOVA followed by Bonferroni post test (FIGS. 18 and 19). This shows that the components may be relatively inert and of low toxicity, as well as volatile with little residue. Morphological images further supported the MTS and LDH results, in which no observable difference could be identified between the experimental rinsing agent groups and the control (HBSS, PFD and F6H8) (FIG. 20A-F). In the study with rMC-1 cells, similar result was obtained.

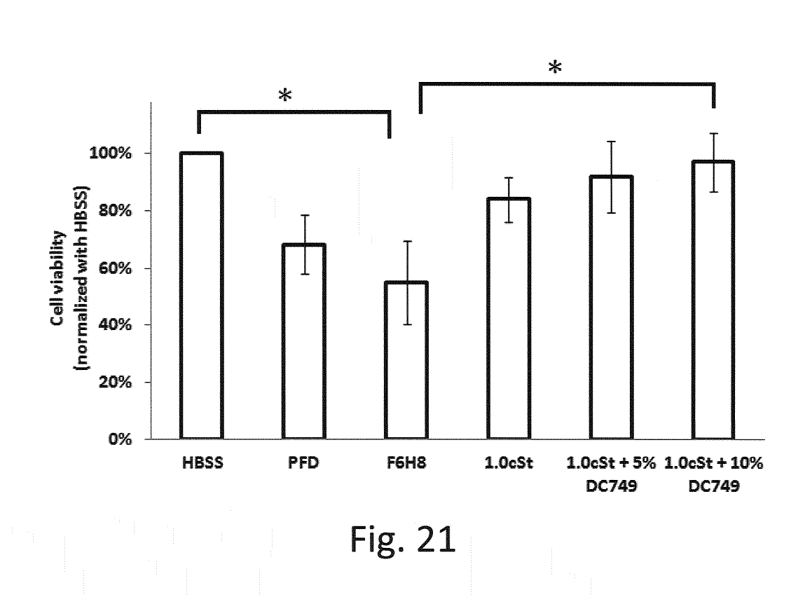

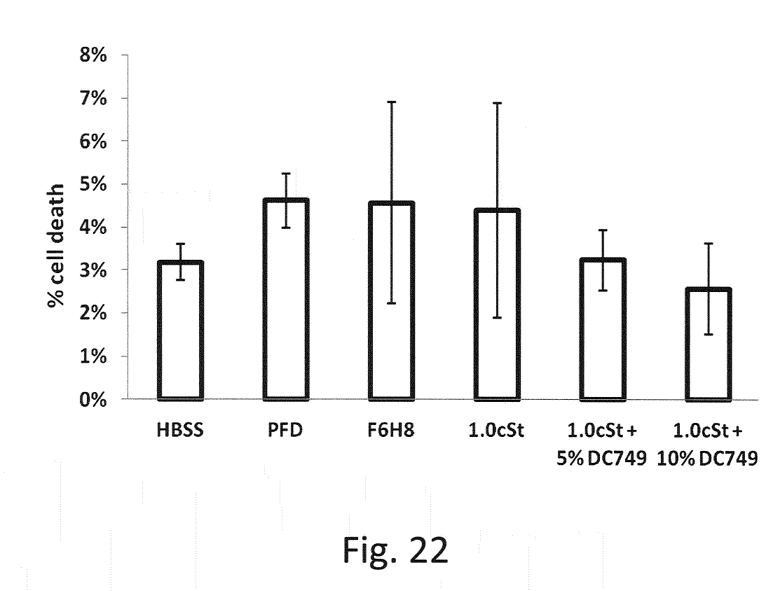

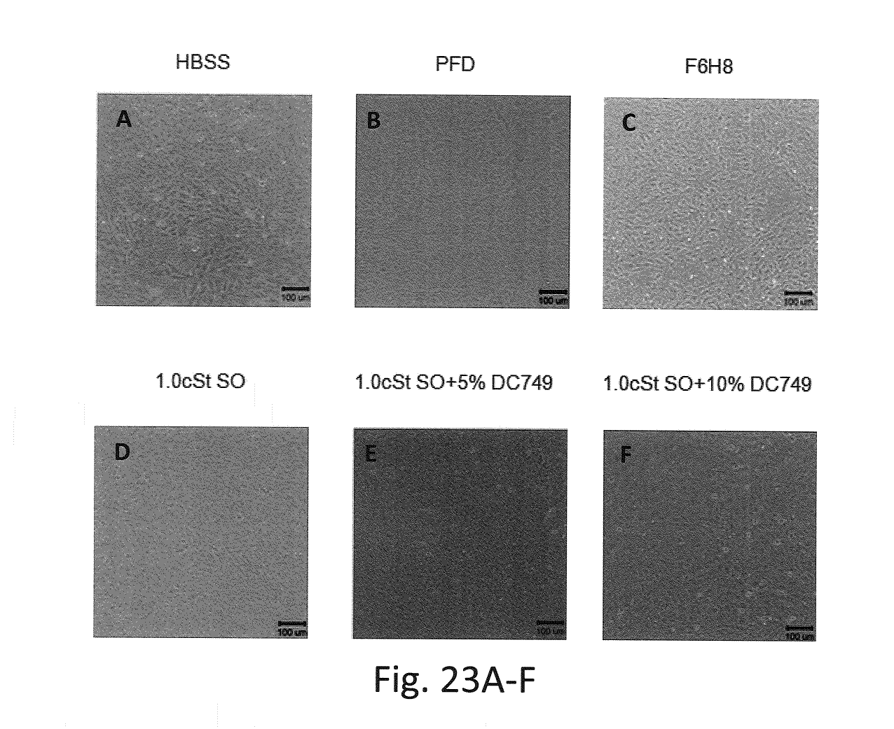

In MTS study, the result showed F6H8 caused a significant decrease in cell viability to the Müller cells than 1.0 cSt+10% DC749. No difference in percentage of dead cell could be observed in all the experimental groups using the proposed rinsing agents with different concentrations of surfactant, when compared with the control, HBSS, and also the references, PFD and F6H8, by one-way ANOVA followed by Bonferroni post test in LDH test (FIGS. 21 and 22). Morphological images supported the MTS and LDH results, in which no observable difference could be identified between the experimental rinsing agent groups and the control (HBSS, PFD and F6H8) (FIGS. 23A-F).

These results suggested that 1.0 cSt silicone-oil-based rinsing agent did not induce cytotoxic effect on both RGC and Muller cells after 1 hr incubation and its cytotoxicity performance was similar to PFD and F6H8, demonstrating a level of biocompatibility that would normally be acceptable by the ophthalmologists.

### 6.5 the Biocompatibility of the Rinsing Agent In Vitro

In a study similar to 6.4 above, the cell viability and the percentage cell death of RGC-5 were investigated in a 30-minute experiment.

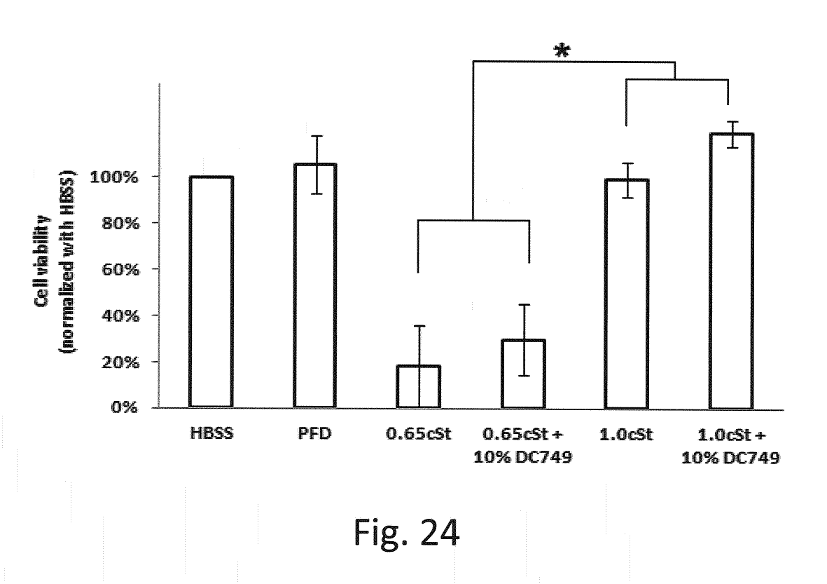

In MTS assay, we did not obtain any statistical difference between the 1.0 mPa·s SO with 10% DC749 and HBSS control and also between the proposed rinse and PFD. Similar result was also obtained in 1.0 mPa·s SO alone when compared with HBSS and PFD. Nevertheless, a significant decrease of cell viability was obtained in 0.65 mPa·s SO and also 0.65 mPa·s SO with 10% DC749 when compared with HBSS control. In addition, significant difference was also obtained between the 0.65 mPa·s SO group and the 1.0 mPa·s SO group (FIG. 24).

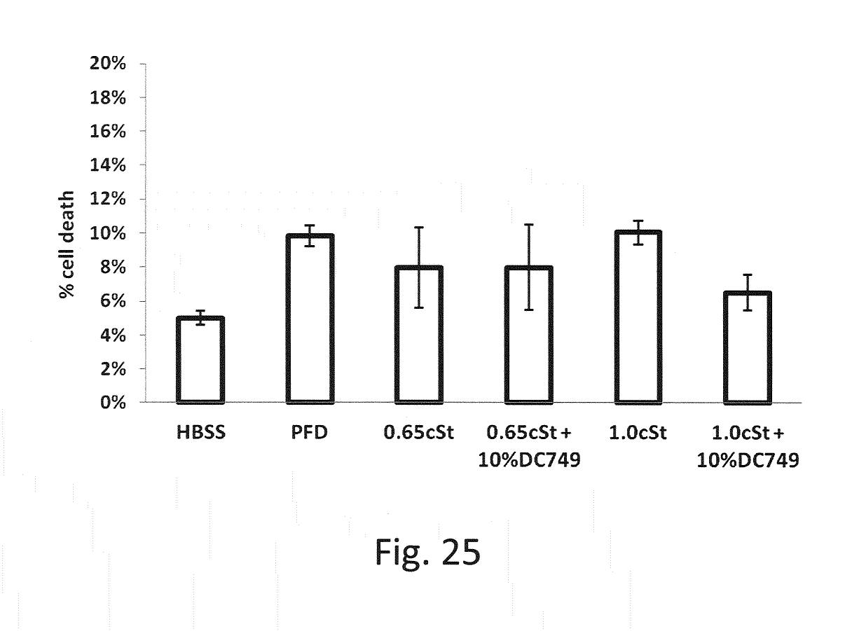

In LDH assay, the percentages of RGC-5 cell death after the exposure of different agents were obtained. There was no significant difference between the HBSS control and the four LMW-SO groups. A lower cell death was observed in 1.0 mPa·s SO+10% DC749 than 1.0 mPa·s SO although this was not statistically significant (FIG. 25).

The results in MTS assay suggested that LMW-SO 0.65 mPa·s had an effect in reducing the metabolism of the RGC-5 cells within 30 minutes but this was not long enough to lead to a significant cell death. The results suggested that LMW-SO 1.0 mPa·s would be a better choice when compared with LMW-SO 0.65 mPa·s for the application.

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

1. A sterile composition comprising (i) a low-molecular-weight silicone oil having a viscosity of 0.5 mPa·s to 10.0 mPa·s, and (ii) a biocompatible hydrophobic surfactant, wherein said composition is 0.5% to 20% v/v of biocompatible hydrophobic surfactant.

2. The composition according to claim 1, wherein the silicone oil has a density of 0.6 g/cm³ to 1.0 g/cm³.

3. The composition according to claim 1, wherein the silicone oil has a density of 0.76 g/cm³ to 0.90 g/cm³.

4. The composition according to claim 1, wherein the silicone oil has a molecular weight of 160 g/mol to 200 g/mol.

5. The composition according to claim 1, wherein the silicone oil has a molecular weight of 200 g/mol to 250 g/mol.

6. The composition according to claim 1, wherein the silicone oil has a surface tension of 15.9 to 18.9 mN/m.

7. The composition according to claim 1, wherein the silicone oil has a viscosity of 1.0 mPa·s to 5.0 mPa·s.

8. The composition according to claim 1, wherein the silicone oil has a viscosity of 5.0 mPa·s to 10.0 mPa·s.

9. The composition according to claim 1, wherein the silicone oil has a viscosity of 0.65 mPa·s to 3.0 mPa·s.

10. The composition according to claim 1, wherein the silicone oil is a straight-chain siloxane.

11. The composition according to claim 1, wherein the silicone oil is a substituted or unsubstituted polydimethylsiloxane (PDMS).

12. The composition according to claim 1, wherein the composition forms a double emulsion.

13. The composition according to claim 1, wherein the biocompatible hydrophobic surfactant is Dow Corning Fluid DC749.

14. The composition according to claim 1, wherein the composition is 1% to 5% v/v of biocompatible hydrophobic surfactant.

15. The composition according to claim 1, wherein the composition is 5% to 10% v/v of biocompatible hydrophobic surfactant.

16. The composition according to claim 12, wherein the double emulsion is an oil-in-water-in-oil double emulsion.

17. The composition according to claim 12, wherein the double emulsion is an encapsulation of emulsified silicone oil droplets by aqueous globules.

18. A method of rinsing an eye cavity, comprising:
initiating a surgical procedure; and
rinsing the eye cavity with a sterile composition comprising (i) a low-molecular-weight silicone oil having a viscosity of 0.5 mPa·s to 10.0 mPa·s, and (ii) a biocompatible hydrophobic surfactant, wherein said composition is 0.5% to 20% v/v of biocompatible hydrophobic surfactant.

19. The composition according to claim 18, wherein said composition is used as a rinsing agent during one or more surgical operations.

20. The composition according to claim 19, wherein said one or more surgical operations comprises vitreoretinal surgery.

21. The method of claim 18 further comprising:
rinsing at least a portion of said eye cavity with said composition wherein said biocompatible hydrophobic surfactant is 50.0% trimethylsiloxysilicate and 50.0% cyclomethicone (decamethylpenta-cyclosiloxane).

22. The method of claim 21, wherein said biocompatible hydrophobic surfactant is Dow Corning Fluid DC749.

23. The method of claim 18, further comprising the steps of: (i) forming a double emulsion in the eye cavity by mixing said composition with emulsified silicone oil droplets in the eye cavity thereby encapsulating emulsified silicone oil droplets with large aqueous globules; (ii) removing the emulsified silicone oil droplets from the eye cavity; and (iii) filling up the eye cavity with long-acting gas after the composition has been removed from the eye cavity.
